SEQUENCE LISTING (197 sequences)

SEQ ID NO: 1 - DNA, 20 bp, Mus musculus
  misc_feature: Mouse RAB7 cDNA primer
ctgaccaaggaggtgatggt

SEQ ID NO: 2 - DNA, 20 bp, Mus musculus
  misc_feature: Mouse RAB7 cDNA primer
gaacagttctctcactctcc

SEQ ID NO: 3 - DNA, 20 bp, Artificial Sequence
  Forward primer of exon 1, gene ZNF9
gattactggcgtgagccacc

SEQ ID NO: 4 - DNA, 10 bp, Artificial Sequence
  5-intron/exon of exon 1, gene ZNF9
cagccatgag

SEQ ID NO: 5 - DNA, 10 bp, Artificial Sequence
  3-intron/exon of exon 1, gene ZNF9
tagaggtatt

SEQ ID NO: 6 - DNA, 20 bp, Artificial Sequence
  Reverse primer of exon 1, gene ZNF9
ggctagtcagaccagtcttg

SEQ ID NO: 7 - DNA, 20 bp, Artificial Sequence
  Forward primer, exon 2, gene ZNF9
caagactggtctgactagcc

SEQ ID NO: 8 - DNA, 10 bp, Artificial Sequence
  5-intron/exon, exon 2, gene ZNF9
aacaggtttc

SEQ ID NO: 9 - DNA, 10 bp, Artificial Sequence
  3-intron/exon, exon 2, gene ZNF9
ggatggtaag

SEQ ID NO: 10 - DNA, 20 bp, Artificial Sequence
  Reverse primer, exon 2, gene ZNF9
cacagttgcatgtgctcacc

SEQ ID NO: 11 - DNA, 20 bp, Artificial Sequence
  Forward primer, exon 3, gene ZNF9
ggtgagcacatgcaactgtg

SEQ ID NO: 12 - DNA, 10 bp, Artificial Sequence
  5-intron/exon, exon 3, gene ZNF9
tgaagcctgc

SEQ ID NO: 13 - DNA, 10 bp, Artificial Sequence
  3-intron/exon, exon 3, gene ZNF9
tataggtaag

SEQ ID NO: 14 - DNA, 20 bp, Artificial Sequence
  Reverse primer, exon 3, gene ZNF9
gccacaggatatcagggcag

SEQ ID NO: 15 - DNA, 20 bp, Artificial Sequence
  Forward primer, exon 4, gene ZNF9
gcatggtgactgttgctttg

SEQ ID NO: 16 - DNA, 10 bp, Artificial Sequence
  5-intron/exon, exon 4, gene ZNF9
tttaggtgtg

SEQ ID NO: 17 - DNA, 10 bp, Artificial Sequence
  3-intron/exon, exon 4, gene ZNF9
cctaattatt

SEQ ID NO: 18 - DNA, 20 bp, Artificial Sequence
  reverse primer, exon 4, gene ZNF9
gggagttgcctctatctgcc

SEQ ID NO: 19 - DNA, 20 bp, Artificial Sequence
  forward primer, exon 1, gene ABTB1
gtctgatgagcctggcccag

SEQ ID NO: 20 - DNA, 10 bp, Artificial Sequence
  5-intron/exon, exon 1, gene ABTB1
agaccatggg

SEQ ID NO: 21 - DNA, 10 bp, Artificial Sequence
  3-intron/exon, exon 1, gene ABTB1
tgcaggtagc

SEQ ID NO: 22 - DNA, 20 bp, Artificial Sequence
  reverse primer, exon 1, gene ABTB1
ggcaaggaaatggtcacctg

SEQ ID NO: 23 - DNA, 20 bp, Artificial Sequence
  forward primer, exon 2A, gene ABTB1
catgacctgatgacccctgc

SEQ ID NO: 24 - DNA, 10 bp, Artificial Sequence
  5-intron/exon, exon 2A, gene ABTB1
cttagattat

SEQ ID NO: 25 - DNA, 20 bp, Artificial Sequence
  reverse primer, exon 2A, gene ABTB1
gaaaagtcacggacctgagg

SEQ ID NO: 26 - DNA, 20 bp, Artificial Sequence
  forward primer, exon 2B, gene ABTB1
ctggcatatgcctcccagag

SEQ ID NO: 27 - DNA, 20 bp, Artificial Sequence
  reverse primer, exon 2B, gene ABTB1
cgcttgcttcctgaggagcc

SEQ ID NO: 28 - DNA, 20 bp, Artificial Sequence
  forward primer, exon 2C, gene ABTB1
cgcttgcttcctgaggagcc

SEQ ID NO: 29 - DNA, 10 bp, Artificial Sequence
  3-intron/exon, exon 2C, gene ABTB1
cgcaggtaaa

SEQ ID NO: 30 - DNA, 20 bp, Artificial Sequence
  reverse primer, exon 2C, gene ABTB1
gccaggctctgtgaaagaag

SEQ ID NO: 31 - DNA, 20 bp, Artificial Sequence
  forward primer, exon 3,  gene ABTB1
cactggcctccaccttccag

SEQ ID NO: 32 - DNA, 10 bp, Artificial Sequence
  5-intron/exon, exon 3, gene ABTB1
cacaggtgat

SEQ ID NO: 33 - DNA, 10 bp, Artificial Sequence
  3-intron/exon, exon 3, gene ABTB1
actgtgtgag

SEQ ID NO: 34 - DNA, 20 bp, Artificial Sequence
  reverse primer, exon 3, gene ABTB1
cactgcctgcagcctttccc

SEQ ID NO: 35 - DNA, 20 bp, Artificial Sequence
  forward primer, exon 4, gene ABTB1
gagggttgagggtgagtacg

SEQ ID NO: 36 - DNA, 10 bp, Artificial Sequence
  5-intron/exon, exon 4, gene ABTB1
tgtaggaggt

SEQ ID NO: 37 - DNA, 10 bp, Artificial Sequence
  3-intron/exon, exon 4, gene ABTB1
gcagggtgag

SEQ ID NO: 38 - DNA, 20 bp, Artificial Sequence
  reverse primer, exon 4, gene ABTB1
caaagcccttatgcgtgcac

SEQ ID NO: 39 - DNA, 20 bp, Artificial Sequence
  forward primer, exon 5, gene ABTB1
gagacagaggctcaggccag

SEQ ID NO: 40 - DNA, 10 bp, Artificial Sequence
  5-intron/exon, exon 5, gene ABTB1
cccaggggtg

SEQ ID NO: 41 - DNA, 10 bp, Artificial Sequence
  3-intron/exon, exon 5, gene ABTB1
aggaggtaag

SEQ ID NO: 42 - DNA, 20 bp, Artificial Sequence
  reverse primer, exon 5, gene ABTB1
cgatgtgggcagactcctgg

SEQ ID NO: 43 - DNA, 20 bp, Artificial Sequence
  forward primer, exon 6, gene ABTB1
ccaatgcaaccaccccagag

SEQ ID NO: 44 - DNA, 10 bp, Artificial Sequence
  5-intron/exon, exon 6, gene ABTB1
cctagattgg

SEQ ID NO: 45 - DNA, 10 bp, Artificial Sequence
  3-intron/exon, exon 6, gene ABTB1
acgtggtgag

SEQ ID NO: 46 - DNA, 20 bp, Artificial Sequence
  reverse primer, exon 6, gene ABTB1
gcagatgaggacacctatcg

SEQ ID NO: 47 - DNA, 20 bp, Artificial Sequence
  forward primer, exon 7, gene ABTB1
gatcgatgacgtcagggagg

SEQ ID NO: 48 - DNA, 10 bp, Artificial Sequence
  5-intron/exon, exon 7, gene ABTB1
ctcagtcgca

SEQ ID NO: 49 - DNA, 10 bp, Artificial Sequence
  3-intron/exon, exon 7, gene ABTB1
tgtgagcgca

SEQ ID NO: 50 - DNA, 20 bp, Artificial Sequence
  reverse primer, exon 7, gene ABTB1
ccgaggccgatccagttatc

SEQ ID NO: 51 - DNA, 20 bp, Artificial Sequence
  forward primer, exon 1, gene RAB7
ggctgctcagacatttgtgc

SEQ ID NO: 52 - DNA, 10 bp, Artificial Sequence
  5-intron/exon, exon 1, gene RAB7
gaaggatgac

SEQ ID NO: 53 - DNA, 10 bp, Artificial Sequence
  3-intron/exon, exon 1, gene RAB7
gaaggatgac

SEQ ID NO: 54 - DNA, 20 bp, Artificial Sequence
  reverse primer, exon 1, gene RAB7
gaagtggcagcacggacagt

SEQ ID NO: 55 - DNA, 21 bp, Artificial Sequence
  forward primer, exon 2, gene RAB7
gtccttcaggtcaggcagatt

SEQ ID NO: 56 - DNA, 10 bp, Artificial Sequence
  5-intron/exon, exon 2, gene RAB7
ttcagagtcg

SEQ ID NO: 57 - DNA, 10 bp, Artificial Sequence
  3-intron/exon, exon 2, gene RAB7
tgcaggtaag

SEQ ID NO: 58 - DNA, 20 bp, Artificial Sequence
  reverse primer, exon 2, gene RAB7
ctgagtatcagccaattatc

SEQ ID NO: 59 - DNA, 20 bp, Artificial Sequence
  forward primer, exon 3, gene RAB7
gcaccccttgcatacatgct

SEQ ID NO: 60 - DNA, 10 bp, Artificial Sequence
  5-intron/exon, exon 3, gene RAB7
cacagatatg

SEQ ID NO: 61 - DNA, 10 bp, Artificial Sequence
  3-intron/exon, exon 3, gene RAB7
gacaagtaag

SEQ ID NO: 62 - DNA, 19 bp, Artificial Sequence
  reverse primer, exon 3, gene RAB7
gtgagcttagcagagaacc

SEQ ID NO: 63 - DNA, 20 bp, Artificial Sequence
  forward primer, exon 4, gene RAB7
gaggatggagtcagtgctgg

SEQ ID NO: 64 - DNA, 10 bp, Artificial Sequence
  5-intron/exon, exon 4, gene RAB7
ttcaggtggc

SEQ ID NO: 65 - DNA, 10 bp, Artificial Sequence
  3-intron/exon, exon 4, gene RAB7
agcaggtggg

SEQ ID NO: 66 - DNA, 20 bp, Artificial Sequence
  reverse primer, exon 4, gene RAB7
gtcagtggtcaggcatcacc

SEQ ID NO: 67 - DNA, 21 bp, Artificial Sequence
  forward primer, exon 5, gene RAB7
cactctgcccaagcagaagtg

SEQ ID NO: 68 - DNA, 10 bp, Artificial Sequence
  5-intron/exon, exon 5, gene RAB7
tccaggaaac

SEQ ID NO: 69 - DNA, 10 bp, Artificial Sequence
  3-intron/exon, exon 5, gene RAB7
ctgagggggc

SEQ ID NO: 70 - DNA, 20 bp, Artificial Sequence
  reverse primer, exon 5, gene RAB7
ggaagaggagaggggaattg

SEQ ID NO: 71 - DNA, 21 bp, Artificial Sequence
  forward primer STR marker 1
cctcattctgcagcgagatgg

SEQ ID NO: 72 - DNA, 22 bp, Artificial Sequence
  reverse primer STR marker 1
tggacagaggcatgaggaagac

SEQ ID NO: 73 - DNA, 22 bp, Artificial Sequence
  forward primer STR marker 2
gtgcagattcactgctgctaac

SEQ ID NO: 74 - DNA, 21 bp, Artificial Sequence
  reverse primer STR marker 2
gagcgagagagaccactgtat

SEQ ID NO: 75 - DNA, 22 bp, Artificial Sequence
  forward primer STR marker 3
gtttgctcatcttgtacagtgc

SEQ ID NO: 76 - DNA, 21 bp, Artificial Sequence
  reverse primer STR marker 3
ctgcagtccactctggaaaca

SEQ ID NO: 77 - DNA, 21 bp, Artificial Sequence
  forward primer STR marker 4
ccaaaaaccttcagctgagtc

SEQ ID NO: 78 - DNA, 21 bp, Artificial Sequence
  reverse primer STR marker 4
caggacgatatacgtgcacac

SEQ ID NO: 79 - DNA, 21 bp, Artificial Sequence
  forward primer exon 1
ccccagctctagatgatttgg

SEQ ID NO: 80 - DNA, 10 bp, Artificial Sequence
  5-intron/exon, exon 1
aatttatgca

SEQ ID NO: 81 - DNA, 10 bp, Artificial Sequence
  3-intron/exon, exon 1
agcaagtacg

SEQ ID NO: 82 - DNA, 20 bp, Artificial Sequence
  reverse primer, exon 1
ggcaaaggagaagacgtgtc

SEQ ID NO: 83 - DNA, 20 bp, Artificial Sequence
  forward primer, exon 2
ctgccagcatcctctcaatg

SEQ ID NO: 84 - DNA, 10 bp, Artificial Sequence
  5-intron/exon, exon 2
tgtagctttc

SEQ ID NO: 85 - DNA, 10 bp, Artificial Sequence
  3-intron/exon, exon 2
tgaaggtaga

SEQ ID NO: 86 - DNA, 20 bp, Artificial Sequence
  reverse primer, exon 2
gtccttgctgtctcaacagc

SEQ ID NO: 87 - DNA, 21 bp, Artificial Sequence
  forward primer, exon 3
cgtgacacatgcgctcacaag

SEQ ID NO: 88 - DNA, 10 bp, Artificial Sequence
  5-intron/exon, exon 3
cgcagatgca

SEQ ID NO: 89 - DNA, 10 bp, Artificial Sequence
  3-intron/exon, exon 3
acagggtccg

SEQ ID NO: 90 - DNA, 20 bp, Artificial Sequence
  reverse primer, exon 3
ctgctcagtgcttgggtctg

SEQ ID NO: 91 - DNA, 21 bp, Artificial Sequence
  forward primer, exon 4
gctgggtgtcaagtaagcatg

SEQ ID NO: 92 - DNA, 10 bp, Artificial Sequence
  5-intron/exon, exon 4
ttaagatcac

SEQ ID NO: 93 - DNA, 10 bp, Artificial Sequence
  3-intron/exon, exon 4
agcaggtgag

SEQ ID NO: 94 - DNA, 21 bp, Artificial Sequence
  reverse primer, exon 4
gctgtctcaaactctgcatcc

SEQ ID NO: 95 - DNA, 21 bp, Artificial Sequence
  forward primer, exon 5
cagcaagcctcagcatagaag

SEQ ID NO: 96 - DNA, 10 bp, Artificial Sequence
  5-intron/exon, exon 5
aacaggttgt

SEQ ID NO: 97 - DNA, 10 bp, Artificial Sequence
  3-intron/exon, exon 5
tggaggtact

SEQ ID NO: 98 - DNA, 20 bp, Artificial Sequence
  reverse primer, exon 5
ggatctaagtattatgtctg

SEQ ID NO: 99 - DNA, 20 bp, Artificial Sequence
  forward primer, exon 6
gctgcagtgagccatgatcg

SEQ ID NO: 100 - DNA, 10 bp, Artificial Sequence
  5-intron/exon, exon 6
cttagcaata

SEQ ID NO: 101 - DNA, 10 bp, Artificial Sequence
  3-intron/exon, exon 6
cttcggtaat

SEQ ID NO: 102 - DNA, 20 bp, Artificial Sequence
  reverse primer, exon 6
ctgatctcctgcaagctgac

SEQ ID NO: 103 - DNA, 20 bp, Artificial Sequence
  forward primer, exon 7
catcacagtgaccgaaagag

SEQ ID NO: 104 - DNA, 10 bp, Artificial Sequence
  5-intron/exon, exon 7
ttcagttttc

SEQ ID NO: 105 - DNA, 10 bp, Artificial Sequence
  3-intron/exon, exon 7
taaaggtaag

SEQ ID NO: 106 - DNA, 20 bp, Artificial Sequence
  reverse primer, exon 7
cagtatcaatagtgccctag

SEQ ID NO: 107 - DNA, 20 bp, Artificial Sequence
  forward primer, exon 8
gagtgttcagtgtggtgggg

SEQ ID NO: 108 - DNA, 10 bp, Artificial Sequence
  5-intron/exon, exon 8
cacagcagga

SEQ ID NO: 109 - DNA, 10 bp, Artificial Sequence
  3-intron/exon, exon 8
tccaggtaag

SEQ ID NO: 110 - DNA, 21 bp, Artificial Sequence
  reverse primer, exon 8
gttcctccactttggaatggc

SEQ ID NO: 111 - DNA, 20 bp, Artificial Sequence
  forward primer, exon 9
ctcctttccattgtcagctg

SEQ ID NO: 112 - DNA, 10 bp, Artificial Sequence
  5-intron/exon, exon 9
tgtaggacat

SEQ ID NO: 113 - DNA, 10 bp, Artificial Sequence
  3-intron/exon, exon 9
acaaggttga

SEQ ID NO: 114 - DNA, 20 bp, Artificial Sequence
  reverse primer, exon 9
ctgtcgttgtccagcaatac

SEQ ID NO: 115 - DNA, 20 bp, Artificial Sequence
  forward primer, exon 10
cctgagactccataccagac

SEQ ID NO: 116 - DNA, 10 bp, Artificial Sequence
  5-intron/exon, exon 10
tccagcttct

SEQ ID NO: 117 - DNA, 10 bp, Artificial Sequence
  3-intron/exon, exon 10
tcacggtaag

SEQ ID NO: 118 - DNA, 21 bp, Artificial Sequence
  reverse primer, exon 10
ctgtcactcagactgaactgg

SEQ ID NO: 119 - DNA, 21 bp, Artificial Sequence
  forward primer, exon 11
cagcaacgggaagtttctcac

SEQ ID NO: 120 - DNA, 10 bp, Artificial Sequence
  5-intron/exon, exon 11
ttcagcccct

SEQ ID NO: 121 - DNA, 10 bp, Artificial Sequence
  3-intron/exon, exon 11
accaagtaag

SEQ ID NO: 122 - DNA, 20 bp, Artificial Sequence
  reverse primer, exon 11
gtaagctccaccatcagcag

SEQ ID NO: 123 - DNA, 21 bp, Artificial Sequence
  forward primer, exon 12
gttctagattcacccctcaac

SEQ ID NO: 124 - DNA, 10 bp, Artificial Sequence
  5-intron/exon, exon 12
tgtagacaaa

SEQ ID NO: 125 - DNA, 10 bp, Artificial Sequence
  3-intron/exon, exon 12
atcaggtaac

SEQ ID NO: 126 - DNA, 21 bp, Artificial Sequence
  reverse primer, exon 12
ccaagttctaccagaagtgag

SEQ ID NO: 127 - DNA, 22 bp, Artificial Sequence
  forward primer, exon 13
cagtgtgatctgactcccaaag

SEQ ID NO: 128 - DNA, 10 bp, Artificial Sequence
  5-intron/exon, exon 13
tgtagaactt

SEQ ID NO: 129 - DNA, 10 bp, Artificial Sequence
  3-intron/exon, exon 13
gacaagttag

SEQ ID NO: 130 - DNA, 21 bp, Artificial Sequence
  reverse primer, exon 13
tgctgattctatcagacaggc

SEQ ID NO: 131 - DNA, 20 bp, Artificial Sequence
  forward primer, exon 14
gctgattctatcagacaggc

SEQ ID NO: 132 - DNA, 10 bp, Artificial Sequence
  5-intron/exon, exon 14
ggcagatctg

SEQ ID NO: 133 - DNA, 10 bp, Artificial Sequence
  3-intron/exon, exon 14
cctaggtaag

SEQ ID NO: 134 - DNA, 20 bp, Artificial Sequence
  reverse primer, exon 14
gtgaggtcagggttgagaag

SEQ ID NO: 135 - DNA, 21 bp, Artificial Sequence
  forward primer, exon 15
cagaatgccagaaacttcccc

SEQ ID NO: 136 - DNA, 10 bp, Artificial Sequence
  5-intron/exon, exon 15
tgcagaagag

SEQ ID NO: 137 - DNA, 10 bp, Artificial Sequence
  3-intron/exon, exon 15
tcaaggtgaa

SEQ ID NO: 138 - DNA, 21 bp, Artificial Sequence
  reverse primer, exon 15
gttcaccacagtgaccgcttc

SEQ ID NO: 139 - DNA, 21 bp, Artificial Sequence
  forward primer, exon 16
gtagtctaggagcctcttagc

SEQ ID NO: 140 - DNA, 10 bp, Artificial Sequence
  5-intron/exon, exon 16
tttagattga

SEQ ID NO: 141 - DNA, 10 bp, Artificial Sequence
  3-intron/exon, exon 16
tgtgggtaag

SEQ ID NO: 142 - DNA, 21 bp, Artificial Sequence
  reverse primer, exon 16
ttgctcagtagagagttggcg

SEQ ID NO: 143 - DNA, 22 bp, Artificial Sequence
  forward primer, exon 17
gaaaatcgaagcttgtcgtggc

SEQ ID NO: 144 - DNA, 10 bp, Artificial Sequence
  5-intron/exon, exon 17
ttcagatcgg

SEQ ID NO: 145 - DNA, 10 bp, Artificial Sequence
  3-intron/exon, exon 17
ggaaggtagg

SEQ ID NO: 146 - DNA, 22 bp, Artificial Sequence
  reverse primer, exon 17
gagtccttgactttgactcagg

SEQ ID NO: 147 - DNA, 20 bp, Artificial Sequence
  forward primer, exon 18
gcaagtgtccctaggaatgg

SEQ ID NO: 148 - DNA, 10 bp, Artificial Sequence
  5-intron/exon, exon 18
taaagcattt

SEQ ID NO: 149 - DNA, 10 bp, Artificial Sequence
  3-intron/exon, exon 18
cccaggtgag

SEQ ID NO: 150 - DNA, 20 bp, Artificial Sequence
  reverse primer, exon 18
gactgtgcatccggtttagc

SEQ ID NO: 151 - DNA, 21 bp, Artificial Sequence
  forward primer, exon 19
cattacaggtgatccttcggg

SEQ ID NO: 152 - DNA, 10 bp, Artificial Sequence
  5-intron/exon, exon 19
ttaagatgga

SEQ ID NO: 153 - DNA, 10 bp, Artificial Sequence
  3-intron/exon, exon 19
tagaggtaag

SEQ ID NO: 154 - DNA, 19 bp, Artificial Sequence
  reverse primer, exon 19
gtgccgaagtcaaggcttc

SEQ ID NO: 155 - DNA, 20 bp, Artificial Sequence
  forward primer, exon 20
ctactgtgttgcagccactg

SEQ ID NO: 156 - DNA, 10 bp, Artificial Sequence
  5-intron/exon, exon 20
cacagggtca

SEQ ID NO: 157 - DNA, 10 bp, Artificial Sequence
  3-intron/exon, exon 20
gccaggtaag

SEQ ID NO: 158 - DNA, 20 bp, Artificial Sequence
  reverse primer, exon 20
cttagcgcttcccagtgcag

SEQ ID NO: 159 - DNA, 20 bp, Artificial Sequence
  forward primer, exon 21
gatttggtggtggcacgaca

SEQ ID NO: 160 - DNA, 10 bp, Artificial Sequence
  5-intron/exon, exon 21
cccagggcac

SEQ ID NO: 161 - DNA, 10 bp, Artificial Sequence
  3-intron/exon, exon 21
gaccggtgag

SEQ ID NO: 162 - DNA, 19 bp, Artificial Sequence
  reverse primer, exon 21
ctcgtggagcatagcagtg

SEQ ID NO: 163 - DNA, 20 bp, Artificial Sequence
  forward primer, exon 22A
ggaatgcgttggggttaagc

SEQ ID NO: 164 - DNA, 10 bp, Artificial Sequence
  5-intron/exon, exon 22A
ttcaggaaga

SEQ ID NO: 165 - DNA, 20 bp, Artificial Sequence
  reverse primer, exon 22A
ctgagcttgtctcacggctc

SEQ ID NO: 166 - DNA, 21 bp, Artificial Sequence
  forward primer, exon 22B
gagtggaggagctggttcatc

SEQ ID NO: 167 - DNA, 10 bp, Artificial Sequence
  3-intron/exon, exon 22B
tataagcagg

SEQ ID NO: 168 - DNA, 20 bp, Artificial Sequence
  reverse primer, exon 22B
gctgtgtctacactggttgg

SEQ ID NO: 169 - DNA, 624 bp, Homo sapiens
  misc_feature: RAB7
atgacctctaggaagaaagtgttgctgaaggttatcatcctgggagattctggagtcgggaagacatcactcatgaaccagtatgtgaataagaaattcagcaatcagtacaaagccacaataggagctgactttctgaccaaggaggtgatggtggatgacaggctagtcacaatgcagatatgggacacagcaggacaggaacggttccagtctctcggtgtggccttctacagaggtgcagactgctgcgttctggtatttgatgtgactgcccccaacacattcaaaaccctagatagctggagagatgagtttctcatccaggccagtccccgagatcctgaaaacttcccatttgttgtgttgggaaacaagattgacctcgaaaacagacaagtggccacaaagcgggcacaggcctggtgctacagcaaaaacaacattccctactttgagaccagtgccaaggaggccatcaacgtggagcaggcgttccagacgattgcacggaatgcacttaagcaggaaacggaggtggagctgtacaacgaatttcctgaacctatcaaactggacaagaatgaccgggccaaggcctcggcagaaagctgcagttgctga

SEQ ID NO: 170 - protein, 207 aa, Homo sapiens
  misc_feature: RAB7
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 171 - DNA, 3366 bp, Homo sapiens
  misc_feature: ARHGEF10
atgcactcagatgaaatgatttatgatgatgttgagaatggggatgaaggtggaaacagctccttggaatacggatggagttcgagtgaatttgaaagttacgaagagcagagtgactcggagtgcaagaatgggattcccaggtccttcctgcgcagcaaccacaaaaagcaactttctcatgacctaacccgtttaaaggagcactatgagaaaaagatgagagatttgatggcaagcacggtgggcgtggtggagattcagcagctcaggcagaagcatgaactgaagatgcagaagctcgtgaaggccgcgaaggacggcaccaaggacgggctggagaggaccagggcagccgtgaagaggggccgctccttcatcaggaccaagtctctcatcgcacaggatcacagatcttctcttgaggaagaacagaatttgttcattgatgttgactgcaagcacccggaagccatcttgaccccgatgcccgagggtttatctcagcagcaggttgtaagaagatatatactgggttcagttgtcgacagtgaaaagaactacgtagatgctcttaagaggattttggagcaatatgagaagccgctgtctgagatggagccaaaggttctgagtgagaggaagctgaagacggtgttctaccgagtcaaagagatcctgcagtgccactcgctatttcagatcgcgctggccagccgcgtttccgagtgggactccgtggaaatgataggcgatgtcttcgtggcttcgttttctaagtccatggtgctggatgcatacagtgaatatgtgaacaatttcagcacagccgtggcagtcctcaagaaaacatgtgccacaaagcccgcttttcttgaatttttaaagcaggaacaggaggccagccccgatcgaaccacgctctacagcctgatgatgaagcccatccagaggttcccacagttcatcctcctgctccaggacatgctgaagaacacctccaaaggccaccccgacaggctgcctcttcagatggccctgacagagctcgaaacactagcagagaagttaaatgaaagaaagagagatgctgatcaacgctgtgaagtgaagcaaatagccaaagccataaacgaaagatacctgaacaagcttctcagcagtggaagccgatacctcattcgatcagatgatatgatagaaacagtttacaacgacagaggagagattgttaaaaccaaagaacgccgagtcttcatgttaaatgatgtgttaatgtgtgccaccgtcagctcacgcccctctcatgacagccgtgtgatgagcagccagaggtacttgctgaagtggagcgttccactgggacatgtggacgccatcgagtatggcagcagcgcaggcacgggcgagcacagcaggcaccttgccgttcacccgccggagagcctggccgtggttgctaacgcgaaaccaaacaaagtttacatggggccaggacaactgtatcaagatttacaaaacttgttgcatgacttaaatgtaattggccaaatcactcagctgataggaaaccttaaaggaaactatcagaacttaaaccagtcagtagcccatgactggacatcaggtttacaaaggcttattttgaagaaagaagatgaaatcagagctgcggactgctgcagaattcagttacagcttcccgggaagcaggacaaatctgggcgaccgacgttctttacagctgtgttcaatacgttcacccctgccatcaaggagtcctgggtcaacagcttacagatggccaagctcgccctagaagaggagaaccacatgggctggttctgtgtggaagacgatgggaatcacattaaaaaggagaagcatcctctcctcgtcggacacatgcccgtgatggtggccaagcagcaggagttcaagattgaatgtgctgcttataaccctgaaccttacctaaataatgaaagccagccagattcattttccacggcacatggtttcctgtggatcggaagttgcacccatcaaatgggtcagattgccatcgtctcgtttcaaaattccactcccaaagtcattgagtgcttcaacgtggaatctcgcatcctgtgcatgctgtacgttcccgtcgaggagaagcgcagagagcctggggcacccccggaccccgagaccccggccgtgagagcttctgatgtccccacgatctgtgtagggacggaggagggaagcatttccatttataaaagcagtcaaggctccaagaaagtgagacttcagcactttttcactcctgagaagtccacagtcatgagcctggcttgcacgtctcagagcctgtacgctggcctggtcaacggggcagtcgccagctacgccagagccccagatggatcctgggattcagaacctcaaaaagtgatcaagttaggcgtcctaccagttagaagtctactcatgatggaagacacgttgtgggcggcttccggaggtcaagtcttcatcatcagtgtggagactcatgctgtagagggtcagctggaggcccaccaggaggaaggcatggtgatctcccacatggccgtgtccggcgtcgggatctggattgccttcacctcagggtccacgctccgcctttttcacacggaaactctcaagcacctgcaggacatcaacatcgccacccctgttcacaacatgctgccagggcaccagcggctgtcggtgacgagcctgctcgtctgccacggattgctgatggtcggcaccagcctgggagtcctcgtggccctgccggtcccacgtctgcaagggattcccaaagtgaccggaagaggcatggtctcctaccatgcacacaacagtcctgtcaaattcatcgtcctggccacggctctgcacgagaaagacaaggacaaatccagggacagcctggctcctggccccgagcctcaggacgaagaccagaaggacgcacttccgagtggaggagctggttcatctctgagccagggtgaccctgacgcagccatctggttgggagattcgctgggatcgatgactcagaaaagcgacctgtcctcctcatctgggtccctgagcttgtctcacggctccagctctctagagcacagatcagaggacagcaccatctatgatctcctgaaggatcctgtctcgctgagaagcaaagcacgccgggccaagaaagccaaggccagctcggcgctggtggtctgtggagggcagggccaccgccgggtgcacaggaaggcccggcagccccaccaggaagagctggcgccgaccgtcatggtctggcagatccctctgctgaatatataa

SEQ ID NO: 172 - protein, 1121 aa, Homo sapiens
  misc_feature: ARHGEF10
MHSDEMIYDDVENGDEGGNSSLEYGWSSSEFESYEEQSDSECKNGIPRSFLRSNHKKQLSHDLTRLKEHYEKKMRDLMASTVGVVEIQQLRQKHELKMQKLVKAAKDGTKDGLERTRAAVKRGRSFIRTKSLIAQDHRSSLEEEQNLFIDVDCKHPEAILTPMPEGLSQQQVVRRYILGSVVDSEKNYVDALKRILEQYEKPLSEMEPKVLSERKLKTVFYRVKEILQCHSLFQIALASRVSEWDSVEMIGDVFVASFSKSMVLDAYSEYVNNFSTAVAVLKKTCATKPAFLEFLKQEQEASPDRTTLYSLMMKPIQRFPQFILLLQDMLKNTSKGHPDRLPLQMALTELETLAEKLNERKRDADQRCEVKQIAKAINERYLNKLLSSGSRYLIRSDDMIETVYNDRGEIVKTKERRVFMLNDVLMCATVSSRPSHDSRVMSSQRYLLKWSVPLGHVDAIEYGSSAGTGEHSRHLAVHPPESLAVVANAKPNKVYMGPGQLYQDLQNLLHDLNVIGQITQLIGNLKGNYQNLNQSVAHDWTSGLQRLILKKEDEIRAADCCRIQLQLPGKQDKSGRPTFFTAVFNTFTPAIKESWVNSLQMAKLALEEENHMGWFCVEDDGNHIKKEKHPLLVGHMPVMVAKQQEFKIECAAYNPEPYLNNESQPDSFSTAHGFLWIGSCTHQMGQIAIVSFQNSTPKVIECFNVESRILCMLYVPVEEKRREPGAPPDPETPAVRASDVPTICVGTEEGSISIYKSSQGSKKVRLQHFFTPEKSTVMSLACTSQSLYAGLVNGAVASYARAPDGSWDSEPQKVIKLGVLPVRSLLMMEDTLWAASGGQVFIISVETHAVEGQLEAHQEEGMVISHMAVSGVGIWIAFTSGSTLRLFHTETLKHLQDINIATPVHNMLPGHQRLSVTSLLVCHGLLMVGTSLGVLVALPVPRLQGIPKVTGRGMVSYHAHNSPVKFIVLATALHEKDKDKSRDSLAPGPEPQDEDQKDALPSGGAGSSLSQGDPDAAIWLGDSLGSMTQKSDLSSSSGSLSLSHGSSSLEHRSEDSTIYDLLKDPVSLRSKARRAKKAKASSALVVCGGQGHRRVHRKARQPHQEELAPTVMVWQIPLLNI

SEQ ID NO: 173 - DNA, 18 bp, Homo sapiens
  misc_feature: c.385C>T sequence variation of RAB7
agattgacntcgaaaaca

SEQ ID NO: 174 - DNA, 18 bp, Homo sapiens
  misc_feature: Wildtype
agattgacctcgaaaaca

SEQ ID NO: 175 - DNA, 18 bp, Homo sapiens
  misc_feature: c.484G>A sequence variation of RAB7
ccatcaacntggagcagg

SEQ ID NO: 176 - DNA, 18 bp, Homo sapiens
  misc_feature: Wildtype
ccatcaacgtggagcagg

SEQ ID NO: 177 - protein, 71 aa, Homo sapiens
  MISC_FEATURE: Rab7 ortholog
ASPRDPENFPFVVLGNKIDLENRQVATKRAQAWCYSKNNIPYFETSAKEAINVEQAFQTIARNALKQETEV

SEQ ID NO: 178 - protein, 71 aa, Mus musculus
  MISC_FEATURE: Rab7 ortholog
ASPRDPENFPFVVLGNKIDLENRQVATKRAQAWCYRKNNIPYFETSAKEAINVEQAFQTIARNALKQETEV

SEQ ID NO: 179 - protein, 71 aa, Rattus norvegicus; identical to SEQ ID NO: 177
  MISC_FEATURE: Rab7 ortholog

SEQ ID NO: 180 - protein, 71 aa, Drosophila melanogaster
  MISC_FEATURE: Rab7 ortholog
ASPRDPDHFPFVVLGNKVDLDNRQVSTRRAQQWCQSKNDIPYYETSAKEGINVEMAFQVIAKNALELEAEA

SEQ ID NO: 181 - protein, 71 aa, Dictyostelium discoideum
  MISC_FEATURE: Rab7 ortholog
AGPRDPDNFPFVVLGNKIDLENQRVVSQKRAASWCQSKGNIPYFETSAKEAINVEQAFQTIARNAIKLEDG

SEQ ID NO: 182 - protein, 72 aa, Caenorhabditis elegans
  MISC_FEATURE: Rab7 ortholog
ASPRDPDHFPFVLLGNKVDLESQRAVSSKRAQSWCQTKGNIPYYEVSAKEALNVEAAFLAIARDALARESQE

SEQ ID NO: 183 - protein, 74 aa, Arabidopsis thaliana
  MISC_FEATURE: Rab7 ortholog
ASPRDPMAFPFILLGNKVDIDGGNSRVVSEKKAREWCAEKGNIVYFETSAKEDYNVDDSFLCITKLALANERDQ

SEQ ID NO: 184 - protein, 73 aa, Saccharomyces cerevisiae
  MISC_FEATURE: Rab7 ortholog
ANVNSPETFPFVILGNKIDAEESKKIVSEKSAQELAKSLGDIPLFLTSAKNAINVDTAFEEIARSALQQNQAD

SEQ ID NO: 185 - DNA, 14 bp, Homo sapiens
  misc_feature: c.326C>T sequence variation of ARHGEF10
gacggcancaagga

SEQ ID NO: 186 - DNA, 14 bp, Homo sapiens
  misc_feature: Control person
gacggcaccaagga

SEQ ID NO: 187 - DNA, 14 bp, Homo sapiens
  misc_feature: c.2111A>G sequence variation of ARGEF10
tgcttcancgtgga

SEQ ID NO: 188 - DNA, 14 bp, Homo sapiens
  misc_feature: Control person
tgcttcaacgtgga

SEQ ID NO: 189 - protein, 40 aa, Homo sapiens
  MISC_FEATURE: GEF10 ortholog
LRQKHELKMQKLVKAAKDGTKDGLERTRAAVKRGRSFIRT

SEQ ID NO: 190 - protein, 40 aa, Mus musculus
  MISC_FEATURE: GEF10 ortholog
AKQRQERKMQKLMKAAKEGTKDGLEKTKAAVKRGGSFIRT

SEQ ID NO: 191 - protein, 40 aa, Fugu rubripes
  MISC_FEATURE: GEF10 ortholog
LKQKHEQKMQKLVKAAKEGTKDGLEKTKAAVKKGRCFIKT

SEQ ID NO: 192 - protein, 28 aa, Macaca fascicularis
  MISC_FEATURE: GEF10 ortholog
MKAAKSGTKDGLEKTRMAVMRKVSFLHR

SEQ ID NO: 193 - protein, 39 aa, Homo sapiens
  MISC_FEATURE: GEF10 ortholog
THQMGQIAIVSFQNSTPKVIECFNVESRILCMLYVPVEE

SEQ ID NO: 194 - protein, 40 aa, Mus musculus
  MISC_FEATURE: GEF10 ortholog
SNQMGQVAIVSFQGSNPKVIECFNVESRILCMVYIPAEES

SEQ ID NO: 195 - protein, 35 aa, Fugu rubripes
  MISC_FEATURE: GEF10 ortholog
QISIVAMQNSSPKVTECFNVESRILCMAYVPVEQP

SEQ ID NO: 196 - protein, 40 aa, Rattus norvegicus
  MISC_FEATURE: GEF10 ortholog
SNQMGQVAIVSFQSANPKVIECFNVESRILCMIYIPAEES

SEQ ID NO: 197 - protein, 7 aa, Macaca fascicularis
  MISC_FEATURE: GEF10 ortholog
VNCPLLE

(19) United States
(12) Patent Application Publication
(10) Pub. No.: US 2005/0208544 A1
(43) Pub. Date: Sep. 22, 2005

(54) Diagnostic tests for the detection of peripheral neuropathy

(76) Inventors: Christine van Broeckhoven, Edegem (BE); Peter de Jonghe, Mechelen (BE); Vincent Timmerman, Broechem (BE); Kristien Verhoeven, Zoersel (BE)

Correspondence Address: TRASK BRITT, P.O. BOX 2550, SALT LAKE CITY, UT, 84110, US

(21) Appl. No.: 11/033,488

(22) Filed: Jan. 10, 2005

Related U.S. Application Data
(63) Continuation of application No. PCT/EP03/50290, filed on Jul. 8, 2003.

(30) Foreign Application Priority Data
Jul. 9, 2002 (EP) 02077724.9
Apr. 8, 2003 (EP) 03076033.4

(51) Int. Cl. C12Q 1/68; C07H 21/04; C07K 14/705; C12N 15/09

(52) U.S. Cl. 435/6; 435/69.1; 435/320.1; 435/325; 530/350; 536/23.5; 536/24.3

(57) ABSTRACT

The present invention relates generally to the field of human genetics. Specifically, the present invention relates to methods and materials used to isolate and detect human peripheral neuropathy causing or predisposing genes, some alleles of which cause peripheral neuropathy.

### CROSS-REFERENCE TO RELATED APPLICATIONS

This application is a continuation of PCT International Patent Application No. PCT/EP2003/050290, filed on Jul. 8, 2003, designating the United States of America, and published, in English, as PCT International Publication No. WO 2004/005541 A1 on Jan. 15, 2004, which claims the benefit under 35 U.S.C. § 119 of European Patent Application Serial No. 03076033.4, filed Apr. 8, 2003 and European Patent Application Serial No. 02077724.9, filed Jul. 9, 2002, the entirety of each of which is incorporated by reference.

### STATEMENT ACCORDING TO 37 C.F.R. § 1.52(e)(5)—SEQUENCE LISTING SUBMITTED ON COMPACT DISC

Pursuant to 37 C.F.R. § 1.52(e)(1)(iii), a compact disc containing an electronic version of the Sequence Listing has been submitted concomitant with this application, the contents of which are hereby incorporated by reference. A second compact disc is submitted and is an identical copy of the first compact disc. The discs are labeled “copy 1” and “copy 2,” respectively, and each disc contains one file entitled “V121.ST25.txt” which is 76 KB and created on Jan. 10, 2005.

### TECHNICAL FIELD

The present invention relates generally to the field of human genetics. Specifically, the present invention relates to methods and materials used to isolate and detect human peripheral neuropathy causing or predisposing genes, some alleles of which cause peripheral neuropathy.

### BACKGROUND OF THE INVENTION

Peripheral neuropathy is a common neurological disorder resulting from damage to the peripheral nerves. It may be acquired and caused by diseases of the nerves or as the result of systemic illness. Many neuropathies have well-defined causes such as diabetes, uremia, AIDS, or nutritional deficiencies. In fact, diabetes is one of the most common causes of peripheral neuropathy. Other causes include mechanical pressure such as compression or entrapment, direct trauma, penetrating injuries, contusions, fracture of dislocated bones, pressure involving the superficial nerves (ulnar, radial, or peroneal), which can result from prolonged use of crutches or staying in one position for too long, or from a tumor, intraneural hemorrhage, exposure to cold or radiation or, rarely, certain medicines or toxic substances, and vascular or collagen disorders, such as atherosclerosis, systemic lupus erythematosus, scleroderma, sarcoidosis, rheumatoid arthritis, and polyarteritis nodosa. In addition, hereditary peripheral neuropathies, among the most common genetic disorders in humans, are a complex, clinically and genetically heterogeneous group of disorders and they produce progressive deterioration of the peripheral nerves. This group of disorders includes hereditary motor and sensory neuropathies (HMSN), hereditary motor neuropathies (HMN) and hereditary sensory neuropathies (HSN). Our understanding of these disorders has progressed from the description of the clinical phenotypes and delineation of the electrophysiologic and pathologic features to the identification of disease genes and elucidation of the underlying molecular mechanisms. Charcot-Marie-Tooth (CMT) disease is the most common inherited disorder of the peripheral nervous system (PNS), with an estimated frequency of 1/2500 individuals. CMT can be divided into two distinct groups based on electrophysiologic studies. CMT type 1 (CMT1) exhibits moderately to severely reduced motor nerve velocity conduction (NCV). The conduction deficit in CMT1 is bilaterally symmetric, which suggests an intrinsic Schwann cell defect. In contrast, CMT type 2 (CMT2) results from neuronal atrophy and degeneration and exhibits normal or only mildly reduced NCV with decreased amplitudes. Recent molecular analysis of the inherited peripheral neuropathies (IPN) has led to important insights into the process of myelination and the function of some of the genes involved. An important problem for the physician is that the IPN show considerable clinical and genetical heterogeneity.¹ The discovery that mutations in multiple genes result in similar phenotypes argues for complex protein interactions and complementing functions for each protein product within the myelin sheath. Knowledge of the structure and function of the causal genes is currently being actively pursued to better classify peripheral neuropathies and to elucidate the underlying molecular mechanisms of these diseases. Thus, the knowledge of the exact genetic aberration in the patients has important ramifications for diagnosis, prognosis, genetic counseling, and approaches for therapy. In the present invention, missense mutations in the small GTPase RAB7 (SEQ ID NOS:169 and 170) and the guanine exchange factor ARHGEF10 (SEQ ID NOS:171 and 172) associated with peripheral neuropathy have been identified. The present invention can be used for the manufacture of a diagnostic assay for a more correct diagnosis of peripheral neuropathies.

### SUMMARY OF THE INVENTION

The present invention relates generally to the field of human genetics. Specifically, the present invention relates to methods and materials used to isolate and detect human peripheral neuropathy causing or predisposing genes (RAB7 and ARHGEF10), some alleles of which cause peripheral neuropathy. In particular, alleles of RAB7 cause Charcot-Marie-Tooth type 2B (CMT2B). Charcot-Marie-Tooth type 2B (CMT2B) (MIM # 600882) or hereditary motor and sensory neuropathy type IIB (HMSN IIB) is clinically characterized by marked distal muscle weakness and wasting, a high frequency of foot ulcers, infections and amputations of the toes due to recurrent infections.² More specifically, the present invention relates to germ line mutations in the RAB7 gene (SEQ ID NO:169) and/or the ARHGEF10 gene (SEQ ID NO:171) and their use in the diagnosis of peripheral neuropathy.

The Rho (Ras homology) proteins have been shown to regulate intracellular signaling activities including organization of the actin cytoskeleton, vesicular trafficking, extension of cellular processes and transcriptional regulation of gene expression. Several studies have demonstrated that the growth cone, by which neurons extend their axons and dendrites towards appropriate targets, is guided by extracellular signals and is transduced via Rho GTPases. The Rho GTPases couple intracellular signal transduction pathways to changes in the external environment. The GTPase has an inactive (GDP-bound) and active (GTP-bound) conformation. Guanine exchange factor proteins (GEFs), such as ARHGEF10, catalyze the release of GDP allowing GTP to bind. In the active GTP-bound state, Rho GTPases interact with target proteins to activate a cellular response. An intrinsic GTPase activity, catalyzed further by GTPase-activating proteins (GAPs), completes the cycle and the GTPase returns to an inactive GDP-bound state. RAB7 belongs to the Rab family of Ras-related GTPases. These Rab proteins are essential for the regulation of intracellular membrane trafficking. The Rab proteins regulate vesicular transport through the recruitment of specific effector or motor proteins and may have a role in linking vesicles and target membranes to the cytoskeleton.^{10, 11}

To date, about 60 human RAB genes have been identified and the majority is likely to control highly specialized functions in many cell types. Mutations in RAB genes may cause a wide range of inherited diseases.¹⁶ RAB7 is involved in the transport between late endosomes and lysosomes and recent studies demonstrate that the RAB7-effector protein RILP (RAB7 interacting lysosomal protein) induces the recruitment of dynein-dynactin motors and regulates transport toward the minus-end of microtubules.^{12, 13} Expression of RAB7-dominant negative mutants in cells inhibits degradation and disperses lysosomes. One such mutant, RAB7N125I, is localized in the GTP-binding domain and proximal to Leu129Phe mutation in families CMT-140 and CMT-126 (FIG. 1, Panel C). In vitro studies demonstrated that this mutant RAB7N125I protein exists preferentially in a nucleotide-free form and has been shown to have a dominant negative effect on late endocytic transport.¹⁴ In contrast, in cells overexpressing RAB7, late endocytic vesicles accumulated in the perinuclear region, probably due to an increased motility in the minus-end direction of microtubules.¹⁵

Thus, the invention discloses methods for determining the presence or absence of RAB7 and/or ARHGEF10 mutations that are useful in the diagnosis or susceptibility to peripheral neuropathy and, more particularly, wherein RAB7 mutations are useful in the diagnosis or susceptibility to CMT2 and, even more particularly, to CMT2B. Mutations of RAB7 (SEQ ID NO:169) causing peripheral neuropathy and, more specifically, CMT2B are included in Table 1A. Mutations of ARHGEF10 (SEQ ID NO:171) causing peripheral neuropathy are included in Table 1B. The amino acid sequence of RAB7 is depicted in SEQ ID NO:170 and the amino acid sequence of ARHGEF10 is depicted in SEQ ID NO:172. These nucleic acids or fragments capable of specifically hybridizing with the corresponding allele in the presence of other RAB7 alleles and/or ARHGEF10 alleles under stringent conditions find broad diagnostic application. Gene products of the disclosed mutant RAB7 and/or ARHGEF10 alleles also find a broad range of diagnostic applications. For example, mutant allelic RAB7 peptides and/or mutant allelic ARHGEF10 peptides can be used to generate specific binding compounds. Binding reagents can be used diagnostically to distinguish wild-type and peripheral neuropathy, more particularly, CMT2B causing RAB7 translation products. The subject nucleic acids (including fragments thereof) may be single or double-stranded and are isolated, partially purified, and/or recombinant. An “isolated” nucleic acid is present as other than a naturally occurring chromosome or transcript in its natural state and isolated from (not joined in sequence to) at least one nucleotide with which it is normally associated on a natural chromosome; a partially pure nucleic acid constitutes at least about 10%, preferably at least about 30%, and more preferably at least about 90% by weight of total nucleic acid present in a given fraction; and a recombinant nucleic acid is joined in sequence to at least one nucleotide with which it is not normally associated on a natural chromosome.

In a first embodiment, the invention provides an isolated nucleic acid coding for a dominant negative, mutant RAB7 polypeptide and/or an isolated nucleic acid coding for a dominant negative, mutant ARHGEF10 polypeptide, the nucleic acid containing, in comparison to the wild-type RAB7 encoding sequence set forth in SEQ ID NO:169, one or more mutations and/or the nucleic acid containing, in comparison to the wild-type ARHGEF10 encoding sequence set forth in SEQ ID NO:171, one or more mutations, wherein the presence of the nucleic acids is indicative for a predisposition or the presence of a peripheral neuropathy.

In yet another embodiment, the invention provides an isolated nucleic acid coding for a dominant negative, mutant RAB7 polypeptide and/or an isolated nucleic acid coding for a dominant negative, mutant ARHGEF10 polypeptide, the nucleic acid containing, in comparison to the wild-type RAB7 encoding sequence set forth in SEQ ID NO:169, one or more mutations selected from the mutations set forth in Table 1A and/or the nucleic acid containing, in comparison to the wild-type ARHGEF10 encoding sequence set forth in SEQ ID NO:171, one or more mutations set forth in Table 1B, wherein the presence of the nucleic acids is indicative for a predisposition or the presence of a peripheral neuropathy.

“Mutant,” as used herein, refers to a gene that encodes a mutant protein. With respect to proteins, the term “mutant” means a protein that does not perform its usual or normal physiological role and that is associated with, or causative of, a pathogenic condition or state. Therefore, as used herein, the term “mutant” is essentially synonymous with the terms “dysfunctional,” “pathogenic,” “disease-causing,” and “deleterious.” With respect to the gene-encoding RAB7 protein and/or ARHGEF10 of the present invention, the term “mutant” refers to a gene encoding RAB7 and/or ARHGEF10, bearing one or more nucleotide/amino acid substitutions, insertions and/or deletions that, for example, can lead to the development of the symptoms of a peripheral neuropathy when expressed in humans. This definition is understood to include the various mutations that naturally exist, including, but not limited to, those disclosed herein, as well as synthetic or recombinant mutations produced by human intervention. The term “mutant,” as applied to the gene encoding RAB7 and/or ARHGEF10, is not intended to embrace sequence variants that, due to the degeneracy of the genetic code, encode proteins identical to the normal sequences disclosed or otherwise presented herein; nor is it intended to embrace sequence variants that, although they encode different proteins, encode proteins that are functionally equivalent to normal RAB7 and/or ARHGEF10. Assays to measure the activity of (mutant) Rab proteins are disclosed in WO 01/20022. These assays can, for example, be used to measure a possible dominant effect of the identified RAB7-mutations in peripheral neuropathy patients. A “dominant negative” allele or a “dominant negative” gene is a mutant allele or mutant gene that, when inherited, manifests the phenotype of the mutation, even in the presence of a wild-type allele or gene.

In another embodiment, the invention provides a nucleic acid probe wherein the nucleotide sequence is a fragment of a nucleic acid sequence derived from a dominant negative, mutant RAB7 gene and/or ARHGEF10 gene.

As used herein, “fragment” refers to a nucleotide sequence of at least about nine nucleotides, typically 15 to 75, or more, wherein the nucleotide sequence comprises at least one mutation for RAB7 and/or ARHGEF10.

In another embodiment, the isolated nucleic acids of the present invention include any of the above-described sequences or fragments of RAB7 and/or ARHGEF10 when included in vectors. Appropriate vectors include cloning vectors and expression vectors of all types, including plasmids, phagemids, cosmids, episomes, and the like, as well as integration vectors. The vectors may also include various marker genes (e.g., antibiotic resistance or susceptibility genes) that are useful in identifying cells successfully transformed therewith. In addition, the vectors may include regulatory sequences to which the nucleic acids of the invention are operably joined and/or may also include coding regions such that the nucleic acids of the invention, when appropriately ligated into the vector, are expressed as fusion proteins. Such vectors may also include vectors for use in yeast “two hybrid,” baculovirus, and phage-display systems. The vectors may be chosen to be useful for prokaryotic, eukaryotic or viral expression, as needed or desired for the particular application. For example, vaccinia virus vectors or simian virus vectors with the SV40 promoter (e.g., pSV2), or Herpes simplex virus or adeno-associated virus may be useful for transfection of mammalian cells including dorsal root ganglia or neurons in culture or in vivo and the baculovirus vectors may be used in transfecting insect cells. A great variety of different vectors are now commercially available and otherwise known in the art and the choice of an appropriate vector is within the ability and discretion of one of ordinary skill in the art.

In yet another embodiment, the invention provides a host cell comprising a recombinant vector according to the invention.

In yet another embodiment, the invention provides a method for the preparation of a diagnostic assay to detect the presence of a peripheral neuropathy in a human comprising detecting at least one mutation in the nucleotide position of SEQ ID NO:169 and/or SEQ ID NO:171 in a tissue sample of the human, wherein the mutation respectively results in a dominant mutation of RAB7 and/or a dominant mutation in ARHGEF10 and wherein the presence of the mutation is indicative of a predisposition or the presence of a peripheral neuropathy.

In yet another embodiment, the invention provides a diagnostic method for determining if a subject bears a mutant gene encoding RAB7 and/or ARHGEF10 comprising the steps of (1) providing a biological sample of the subject and (2) detecting in the sample a mutant nucleic acid encoding a RAB7 protein and/or ARHGEF10 or a mutant RAB7 protein activity and/or a mutant ARHGEF10 activity.

The RAB7 and/or ARHGEF10 gene and gene product, as well as other products derived thereof (e.g., probes, antibodies), can be useful in the diagnosis of peripheral neuropathy and probably also in acquired forms of peripheral neuropathy (in other words, to detect if a human has a predisposition to acquire a peripheral neuropathy or, more particularly, CMT2B in the case of RAB7). Diagnosis of, for example, inherited cases of these diseases can be accomplished by methods based upon the nucleic acids (including genomic and mRNA/cDNA sequences), proteins, and/or antibodies. Preferably, the methods and products are based upon the human RAB7 and/or ARHGEF10 gene, protein or antibodies against the RAB7 and/or ARHGEF10 protein. As will be obvious to one of ordinary skill in the art, however, the significant evolutionary conservation of large portions of RAB7 and/or ARHGEF10 nucleotide and amino acid sequences, even in species as diverse as humans and C. elegans and Drosophila, allow the skilled artisan to make use of such non-human RAB7- and/or ARHGEF10-homologue nucleic acids, proteins and antibodies, even for applications directed toward human or other mammalian subjects. Thus, for brevity of exposition, but without limiting the scope of the invention, the following description will focus upon uses of the human homologues of RAB7 and/or ARHGEF10 genes and proteins. It will be understood, however, that homologous sequences from other species will be equivalent for many purposes. As will be appreciated by one of ordinary skill in the art, the choice of diagnostic methods of the present invention will be influenced by the nature of the available biological samples to be tested and the nature of the information required. The RAB7 and/or ARHGEF10 gene is highly expressed in dorsal root ganglia (sensory neurons) and the ventral horn (motor neurons), but motor neuron biopsies are invasive, dangerous, difficult and expensive procedures, particularly for routine screening. Other tissues which express the RAB7 and/or ARHGEF10 gene at significant levels, however, may demonstrate alternative splicing (e.g., white blood cells) and, therefore, mRNA derived from the RAB7 gene or proteins from such cells may be less informative. Thus, assays based upon a subject's genomic DNA may be the preferred methods for diagnostics of the RAB7 and/or ARHGEF10 gene as no information will be lost due to alternative splicing and because essentially any nucleate cells may provide a usable sample. When the diagnostic assay is to be based upon nucleic acids from a sample, either mRNA or genomic DNA may be used. When mRNA is used from a sample, many of the same considerations apply with respect to source tissues and the possibility of alternative splicing. That is, there may be little or no expression of transcripts unless appropriate tissue sources are chosen or available, and alternative splicing may result in the loss of some information. With either mRNA or DNA, standard methods well known in the art may be used to detect the presence of a particular sequence, either in situ or in vitro (see, e.g. Genome Analysis, A laboratory Manual, eds E. D. Green, B. Birren, S. Klapholz, R. M. Myers, P. Hieter, Cold Spring Harbor Laboratory Press, 1997). In a preferred embodiment of the invention, the starting nucleic acid represents a sample of DNA isolated from an animal or human patient. This DNA may be obtained from any cell source or body fluid. Non-limiting examples of cell sources available in clinical practice include blood cells, buccal cells, cervico-vaginal cells, epithelial cells from urine, or any cells present in tissue obtained by biopsy. Body fluids include blood, urine, and cerebrospinal fluid. DNA is extracted from the cell source or body fluid using any of the numerous methods that are standard in the art. It will be understood that the particular method used to extract DNA will be chosen as being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction and/or phenol extractions can be used to obtain nucleic acid from cells or tissues, e.g., blood. In a specific embodiment, the cells may be directly used without purification of the target nucleic acid. For example, the cells can be suspended in hypotonic buffer and heated to about 90-100° C., until cell lysis and dispersion of intracellular components occur, generally about 1 to 15 minutes. After the heating step, the amplification reagents may be added directly to the lysed cells. This direct amplification method may, for example, be used on peripheral blood lymphocytes. The preferred amount of DNA to be extracted for analysis of human genomic DNA is at least 5 picograms (corresponding to about 1 cell equivalent of a genome size of 4×10⁹ base pairs). In some applications, such as, for example, detection of sequence alterations in the genome of a microorganism, variable amounts of DNA may be extracted.

In a particular embodiment, the starting nucleic acid is RNA obtained, e.g., from a cell or tissue. RNA can be obtained from a cell or tissue according to various methods known in the art and described, e.g., Genome Analysis, A laboratory Manual, eds E. D. Green, B. Birren, S. Klapholz, R. M. Myers, P. Hieter, Cold Spring Harbor Laboratory Press, 1997. For in situ detection of a mutant nucleic acid sequence of RAB7 and/or ARHGEF10, a sample of tissue may be prepared by standard techniques and then contacted with a probe, preferably one which is labeled to facilitate detection, and an assay for nucleic acid hybridization is conducted under stringent conditions which permit hybridization only between the probe and highly or perfectly complementary sequences. In many applications, the nucleic acids are labeled with directly or indirectly detectable signals or means for amplifying a detectable signal. Examples include radiolabels, luminescent (e.g., fluorescent) tags, components of amplified tags, such as antigen-labeled antibody, biotin-avidin combinations, etc. The nucleic acids can be subject to purification, synthesis, modification, sequencing, recombination, incorporation into a variety of vectors, expression, transfection, administration or methods of use disclosed in standard manuals such as Genome Analysis, A laboratory Manual, eds E. D. Green, B. Birren, S. Klapholz, R. M. Myers, P. Hieter, Cold Spring Harbor Laboratory Press, 1997, or that are otherwise known in the art. Because many mutations in genes that cause diseases detected to date consist of a single nucleotide substitution, high stringency hybridization conditions will be required to distinguish normal sequences from most mutant sequences. A significant advantage of the use of either DNA or mRNA is the ability to amplify the amount of genetic material using the polymerase chain reaction (PCR), either alone (with genomic DNA) or in combination with reverse transcription (with mRNA to produce cDNA). Other nucleotide sequence amplification techniques may be used, such as ligation-mediated PCR, anchored PCR and enzymatic amplification as will be understood by those skilled in the art. Sequence alterations may also generate fortuitous restriction enzyme recognition sites which are revealed by the use of appropriate enzyme digestion followed by gel-blot hybridization. DNA fragments carrying the site (normal or mutant) are detected by their increase or reduction in size, or by the increase or decrease of corresponding restriction fragment numbers. Genomic DNA samples may also be amplified by PCR prior to treatment with the appropriate restriction enzyme and the fragments of different sizes are visualized, for example, under UV light in the presence of ethidium bromide, after gel electrophoresis. Genetic testing based on DNA sequence differences may be achieved by detection of alteration in electrophoretic mobility of DNA fragments in gels. Small sequence deletions and insertions can be visualized by high resolution gel electrophoresis of single-stranded DNA, or as changes in the migration pattern of DNA heteroduplexes in non-denaturing gel electrophoresis. Alternatively, a single base substitution mutation may be detected based on differential PCR product length in PCR. The PCR products of the normal and mutant gene may be differentially detected in acrylamide gels. Nuclease protection assays (S1 or ligase-mediated) also reveal sequence changes at specific locations or, alternatively, to confirm or detect a polymorphism resulting in restriction mapping changes. Ligated PCR, allele-specific oligonucleotide probes (ASOs), REF-SSCP chemical cleavage, endonuclease cleavage at mismatch sites or SSCP may be used. Both REF-SSCP and SSCP are mobility shift assays which are based upon the change in conformation due to mutations. DNA fragments may also be visualized by methods in which the individual DNA samples are not immobilized on membranes. The probe and target sequences may be in solution or the probe sequence may be immobilized. Autoradiography, radioactive decay, spectrophotometry and fluorometry may also be used to identify specific individual genotypes. Mutations in RAB7 and/or ARHGEF10 can also be detected by direct nucleotide sequencing. Methods for nucleotide sequencing are well known in the art. Fragments of the disclosed alleles of RAB7 and/or ARHGEF10 are sufficiently long for use as specific hybridization probes for detecting endogenous alleles and, particularly, to distinguish the disclosed mutant alleles which correlate with peripheral neuropathy, more particularly, CMT2B alleles in the use of RAB7. Preferred fragments are capable of hybridizing to the corresponding mutant allele under stringency conditions characterized by a specific hybridization buffer. In any event, the fragments are necessarily of length sufficient to be unique to the corresponding allele; i.e., has a nucleotide sequence at least long enough to define a novel oligonucleotide, usually at least about 14, 16, 18, 20, 22, or 24 bp in length, though such fragment may be joined in sequence to other nucleotides which may be nucleotides which naturally flank the fragment. For example, where the subject nucleic acids are used as PCR primers or hybridization probes, the subject primer or probe comprises an oligonucleotide complementary to a strand of the mutant or rare allele of length sufficient to selectively hybridize with the mutant or rare allele. Generally, these primers and probes comprise at least 16 bp to 24 bp complementary to the mutant or rare allele and may be as large as is convenient for the hybridization conditions. In some cases where the critical mutation in RAB7 and/or ARHGEF10 is a deletion of wild-type sequence, useful primers/probes require wild-type sequences flanking (both sides) the deletion with at least two, usually at least three, more usually at least four, most usually at least five, bases. Where the mutation is an insertion or substitution which exceeds about 20 bp, it is generally not necessary to include wild-type sequences in the probes/primers. For insertions or substitutions of fewer than 5 bp, preferred nucleic acid portions comprise and flank the substitution/insertion with at least two, preferably at least three, more preferably at least four, most preferably at least five, bases. For substitutions or insertions from about 5 to about 20 bp, it is usually necessary to include both the entire insertion/substitution and at least two, usually at least three, more usually at least four, most usually at least five, bases of wild-type sequence of at least one flank of the substitution/insertion.

The wording “stringent hybridization conditions” is a term of art understood by those of ordinary skill in the art. For any given nucleic acid sequence, stringent hybridization conditions are those conditions of temperature, chaotrophic salts, pH and ionic strength which will permit hybridization of that nucleic acid sequence to its complementary sequence and not to substantially different sequences. The exact conditions which constitute “stringent” conditions, depend upon the nature of the nucleic acid sequence, the length of the sequence, and the frequency of occurrence of subsets of that sequence within other non-identical sequences. By varying hybridization conditions from a level of stringency at which non-specific hybridization conditions occurs to a level at which only specific hybridization is observed, one of ordinary skill in the art can, without undue experimentation, determine conditions which will allow a given sequence to hybridize only with complementary sequences. Hybridization conditions, depending upon the length and commonality of a sequence, may include temperatures of 20° C.-65° C. and ionic strengths from 5× to 0.1×SSC. Highly stringent hybridization conditions may include temperatures as low as 40-42° C. (when denaturants such as formamide are included) or up to 60-65° C. in ionic strengths as low as 0.1×SSC. These ranges, however, are only illustrative and, depending upon the nature of the target sequence and possible future technological developments, may be more stringent than necessary.

In yet another embodiment, the invention provides a method for the preparation of a diagnostic assay to detect the presence of CMT2B in a human comprising detecting at least one mutation in the nucleotide sequence of SEQ ID NO:169 in a tissue sample of the human, wherein the mutation results in a dominant mutation of RAB7 and wherein the presence of the mutation is indicative of a predisposition or the presence of CMT2B.

In yet another embodiment, the invention provides a method for the preparation of a diagnostic assay to detect the presence of a peripheral neuropathy, more particularly CMT2B, in a human comprising detecting at least one mutation in the nucleotide sequence of SEQ ID NO:169 in a tissue sample of the human, wherein the mutation is derived from Table 1A and wherein the presence of the mutation is indicative of a predisposition or the presence of a peripheral neuropathy, more particularly CMT2B.

In yet another embodiment the invention provides a method for the preparation of a diagnostic assay to detect the presence of a peripheral neuropathy, more particularly HMSN, in a human comprising detecting at least one mutation in the nucleotide sequence of SEQ ID NO:171 in a tissue sample of the human, wherein the mutation is derived from Table 1B and wherein the presence of the mutation is indicative of a predisposition or the presence of a peripheral neuropathy, more particularly HMSN.

When a diagnostic assay is to be based upon RAB7 and/or ARHGEF10 proteins, a variety of approaches are possible. For example, diagnosis can be achieved by monitoring differences in the electrophoretic mobility of normal and mutant RAB7 and/or ARHGEF10 protein. Such an approach will be particularly useful in identifying mutants in which charge substitutions are present or in which insertions, deletions or substitutions have resulted in a significant change in the molecular mass of the resultant protein. Alternatively, diagnosis may be based upon differences in the proteolytic cleavage patterns of normal and mutant RAB7 and/or ARHGEF10 protein, differences in molar ratios of the various amino acid residues, or by functional assays demonstrating altered function of the gene products. In some preferred embodiments, protein-based diagnostics will employ differences in the ability of antibodies to bind to normal and mutant RAB7 and/or ARHGEF10 proteins. Such diagnostic tests may employ antibodies which bind to the normal proteins but not to mutant proteins, or vice versa. In particular, an assay in which a plurality of monoclonal antibodies, each capable of binding to a mutant epitope, may be employed. The levels of anti-mutant examples binding in a sample obtained from a test subject (visualized by, for example, radiolabeling, ELISA or chemiluminescence) may be compared to the levels of binding to a control sample. Such antibody diagnostics may be used for in situ immunohistochemistry using biopsy samples of (CNS) tissues obtained ante mortem or post-mortem or may be used with fluid samples, such as cerebrospinal fluid, or with peripheral tissues, such as white blood cells.

In another embodiment, the invention provides a transgenic non-human animal comprising a vector comprising a dominant mutant of RAB7.

In yet another embodiment, the invention provides a transgenic non-human animal comprising a vector comprising a mutation of RAB7 listed in Table 1A.

In a further embodiment, the invention provides a transgenic non-human animal comprising a vector comprising a dominant mutant of ARHGEF10.

In a yet further embodiment, the invention provides a transgenic non-human animal comprising a vector comprising a mutation of ARHGEF10 listed in Table 1B.

### DESCRIPTION OF THE FIGURES

FIG. 1 illustrates the DNA and protein sequence analysis of RAB7. Panel A is an electropherogram showing the C385T (also commonly referred to as c.385C>T and 385C→T) sequence variation in part of exon 3 resulting in the Leu129Phe missense mutation in families CMT-140 and CMT-126, and the isolated patient CMT-186.26 (see SEQ ID NO:173). Panel B is an electropherogram of the G484A (also commonly referred to as c.484G>A and 484G→A) sequence variation in part of exon 4 resulting in the Val162Met missense mutation in families CMT-90 and CMT-195, and the isolated patients CMT-186.28 and PN626.1 (see SEQ ID NO:175). The corresponding genomic sequence of a control person is shown below. Panel C shows clustalW multiple protein alignment (http:H/npsa-pbil.ibcp.fr/cgi-bin/npsa_automat.pl?page=npsa_clustalw.html) of the Rab7 orthologs of the region surrounding the Leu129Phe and Val162Met mutations (SEQ ID NOS:177-184, in order from top to bottom). Rab7 orthologs: Human (H. sapiens), mouse (M. musculus), rat (R. norvegicus), fly (D. melanogaster), slime mold (D. discoideum), nematode (C. elegans), mouse-ear cress (A. thaliana), and baker's yeast (S. cerevisiae). The highly conserved motif involved in guanine nucleotide binding is boxed. Both amino acid mutations are shaded and indicated by an arrow.

FIG. 2 depicts a haplotype analysis of chromosome 8p23 STR markers in family CMT-54 and PN-648. Legend to the symbols: squares=male, circle=female, open=unaffected, filled=affected, slashed=deceased, arrow=recombination event, box=disease-associated haplotype. The best genetic and physical order of STR markers is according to NCBI. Genotypes are represented by allele numbers, and “0-0”=failed genotype. The ARHGEF10 gene is located between markers D8S156 and D8S264. The clinical, neurophysiological and neuropathological findings in family CMT-54 have been reported in detail elsewhere (De Jonghe et al. 1999). In family PN-648, the single patient III-3 is a female of 54 years old. Her mother deceased at 65 years because of breast cancer and her father deceased at 89 years old. No neurological diseases were found in her relatives. At age 4 to 5 years old, her clinical history started with walking disturbances. Two years later, slight distal hypotrophy of legs and arms was noticed. At 8 years, she walked with difficulty and limb distal atrophy was more evident. At 11 years, she was not able to walk unassisted and at 14 and 16 years, she underwent surgical operation on the knee and feet, respectively, due to severe ankylosis. Her clinical picture remained stable from 16 to 30 years and she was able to walk if assisted. At 30 years of age, she developed a dorso-lumbar kypho-scoliosis and 7 years later she became wheel-chair bound. The MRI of brain and spinal cord were normal. At 49 years old, she showed severe distal hypotrophy of arms and legs and in the hands and feet, only bone structures were appreciable. The upper limbs showed important weakness in the proximal muscles; developed severe weakness of forearm and hand (Medical Research Council, MRC score=0). The lower limbs showed important proximal weakness, severe distal weakness, dropping feet, absent deep tendon reflexes and sensory disturbances. Because of the almost complete absence of muscular tissue, it was impossible to perform a neurophysiological study. Electromyography was performed when she was 30 years old; the motor NCVs were not evoked in the right sciatic-popliteus and median nerves, and the sensory NCVs were reduced on the right median nerve. At 49 years old, the sensory and motor NCV were not evoked in the ulnar nerve. Finally, the sural nerve biopsy of patient PN-648 III-3′ showed severe reduction in the density of myelinated fibers (about 85-90% of fibers were lost). The large size fibers were absent. There were no classical onion bulb formations (only some early and simple onion bulbs were observed) and the endoneural connective tissue was increased.

FIG. 3 is a genetic map of the 8p23 chromosomal region. Legend: Map showing the contigs covering the 8p23 region and the distribution of the ten polymorphic markers used for haplotyping family CMT-54. Approximate genetic distances are according to GenBank. The location of the candidate genes, KIAA0711 (hypothetical protein KIAA0711), MYOM2 (myomesin (M-protein) 2), CLN8 (ceroid-lipofuscinosis, neuronal 8), DLGAP2 (discs, large (Drosophila) homolog-associated protein 2) and ARHGEF10 (Rho guanine nucleotide exchange factor 10), screened for mutations are indicated. Arrows define the recombination events in candidate region for CMT-54. D8Skris4 is also designated as STR1, D8Skris9 is also designated as STR2, D8SkrisCA2 is also designated as STR3, D8Skris6 is also designated as STR4).

FIG. 4 depicts mutation analysis of ARHGEF10. Panel A includes electropherograms showing the C326T (also commonly referred to as c.326C>T and 326C→T), sequence variation in exon 3 resulting in the Thr109lle missense mutation in family CMT-54 and the A2111G (also commonly referred to as c.211A>G and 211A→G) sequence variation in exon 17 resulting in the Asn704Ser missense mutation in patient PN-648.1 (see SEQ ID NOS:185 and 187, respectively). The corresponding genomic sequence of a control person is shown in the electropherogram below. Panel B includes clustalW multiple protein alignment of the human (Homo sapiens), mouse (Mus musculus), fugu (Fugu rubripes), rat (Rattus norvegicus) and macaque (Macaca fascicularis) GEF10 orthologues SEQ ID NOS:189-197, in order, from top to bottom). Both amino acid mutations Thr109lle and Asn704Ser are shaded.

### DETAILED DESCRIPTION OF THE INVENTION

Since the isolated RAB7 and/or ARHGEF10 mutations are dominant (dominant negative), an alternative method for constructing a cell line is to engineer a genetically mutated gene, or a portion thereof, into an established (either stably or transiently) cell line of choice. In another embodiment, the present invention provides a transgenic non-human animal that carries in its somatic and germ cells at least one integrated copy of a human DNA sequence that encodes a mutant RAB7 and/or ARHGEF10 protein or fragment thereof. It is expected that the transgenic non-human animal, for example a transgenic mouse, will have a particular value because, likewise in the human CMT2B patients with the same pathogenic mutations in RAB7, a transgenic animal with an axonal phenotype is expected. In a preferred example, it may be possible to excise the mutated RAB7 and/or ARHGEF10 gene for use in the creation of transgenic animals containing the mutated gene. In another example, an entire human RAB7 mutant allele and/or an entire human ARHGEF10 mutant allele may be cloned and isolated, either in parts or as a whole, in a cloning vector (e.g., cosmid or yeast or human artificial chromosome). The human variant RAB7 mutant and/or ARHGEF10 mutant, either in parts or in whole, may be transferred to a host non-human animal, such as a mouse or a rat. As a result of the transfer, the resultant transgenic non-human animal will preferably express one or more mutant RAB7 and/or ARHGEF10 polypeptides. Most preferably, a transgenic non-human animal of the invention will express one or more mutant RAB7 and/or ARHGEF10 polypeptides in a motor neuron-specific manner (e.g., dorsal root ganglia). Alternatively, one may design minigenes encoding mutant RAB7 and/or ARHGEF10 polypeptides. Such mini-genes may contain a cDNA sequence encoding a mutant RAB7 and/or ARHGEF10 polypeptide, preferably full-length, a combination of RAB7 and/or ARHGEF10 gene exons, or a combination thereof, linked to a downstream polyadenylation signal sequence and an upstream promoter (and preferably enhancer). Such a mini-gene construct will, when introduced into an appropriate transgenic host (e.g., mouse or rat), express an encoded mutant RAB7 and/or ARHGEF10 polypeptide.

Another approach to create transgenic animals is to target a mutation to the desired gene by homologous recombination in an embryonic stem (ES) cell line in vitro, followed by microinjection of the modified ES cell line into a host blastocyst and subsequent incubation in a foster mother (see Frohman and Martin (1989) Cell 56:145). Alternatively, the technique of microinjection of the mutated gene, or a portion thereof, into a one-cell embryo followed by incubation in a foster mother can be used. Various uses of transgenic animals are known in the art. Alternatively, site-directed mutagenesis and/or gene conversion can be used to mutate a murine (or other non-human) RAB7 and/or ARHGEF10 gene allele, either endogenous or transfected. The procedure for generating transgenic rats is similar to that of mice (Hammer et al., Cell 63; 1099-112 (1990)). Thirty day-old female rats are given a subcutaneous injection of 20 IU of PMSG (0.1 cc) and 48 hours later, each female placed with a proven male. At the same time, 40-80 day old females are placed in cages with vasectomized males. These will provide the foster mothers for embryo transfer. The next morning, females are checked for vaginal plugs. Females who have mated with vasectomized males are held aside until the time of transfer. Donor females that have mated are sacrificed (CO₂ asphyxiation) and their oviducts removed, placed in DPBS (Dulbecco's phosphate buffered saline) with 0.5% BSA and the embryos collected. Cumulus cells surrounding the embryos are removed with hyaluronidase (1 mg/ml). The embryos are then washed and placed in EBSS (Earle's balanced salt solution) containing 0.5% BSA in a 37.5° C. incubator until the time of microinjection. Once the embryos are injected, the live embryos are moved to DPBS for transfer into foster mothers. The foster mothers are anesthetized with ketamine (40 mg/kg, ip) and xylazine (5 mg/kg, ip). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10-12 embryos are transferred into each rat oviduct through the infundibulum. The incision is then closed with sutures, and the foster mothers are housed singly.

### EXAMPLES

1. RAB7

A molecular genetic study of three families with an ulcero-mutilating phenotype that were previously linked to the CMT2B locus⁷⁻⁹ was performed. To determine whether the American (CMT-195),⁷ Scottish (CMT-90)⁸ and Austrian (CMT-140)⁹ families share a common disease-associated haplotype, 15 STR markers in the CMT2B region were analyzed. These markers included six new polymorphic STR markers (D3SCMT126A, D3SCMT126B, D3SCMT126C, D3SCMT126D, D3SCMT126F and D3SCMT126G) that were isolated by using sequence information from the public databases. For each marker, alleles associated with the ulcero-mutilating phenotype were identified and a disease haplotype was constructed in each family. No common disease haplotype was found in families CMT-195, CMT-90 and CMT-140, suggesting the absence of a genetic relationship between the CMT2B families. However, a common disease haplotype spanning nine STR markers, from D3S3519 to D3SCMT126C, was shared between the Austrian family CMT-140 and a small branch of the Austrian multigenerational pedigree CMT-126 (patients III-5, IV-2, IV-3 and V-1), originally excluded for the CMT2B locus.³ Although the five remaining patients (III-1, III-2, III-3, III-6 and IV-6) with an ulcero-mutilating phenotype in CMT-126 do not have the same disease haplotype, it is highly unlikely that CMT-140 and part of the CMT-126 family share the same alleles over a nine-marker interval by chance.

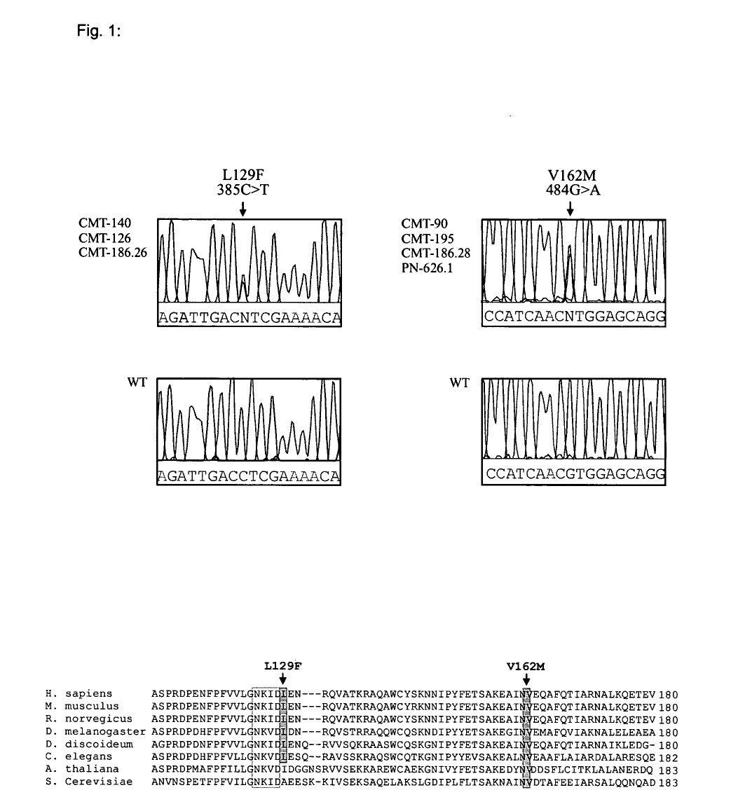

In family CMT-140 and CMT-126, recombination in affected individuals V-10, V-12, V-13 and VI-5 between markers D3S1589 and D3S3584, mapping the CMT2B locus telomeric to D3S1589 was observed. The informative recombination in CMT-126 (III-5, IV-2, IV-3 and V-1) maps the CMT2B locus centromeric to marker D3SCMT126D. These data refine the CMT2B region to 2.5 cM, between D351589 and D3SCMT126D. In the CMT2B locus, three known positional candidate genes for mutation analysis in the CMT2B families were selected: ZNF9 (zinc finger protein 9), ABTB1 (ankyrin repeat and BTB domain containing 1), and RAB7 (small GTPase late endosomal protein RAB7). For each gene, all known exons and intron-exon boundaries in CMT2B patients were sequenced and no disease-causing mutations were found in ZNF9 and ABTB1. However, in exon 3 of RAB7, a C385T mutation (Leu129Phe) in family CMT-140 and in the small branch of family CMT-126 was found (FIG. 1, Panel A). A second G484A mutation (Val162Met) in exon 4 was found in families CMT-195 and CMT-90 (FIG. 1, Panel B). The missense mutations segregate with the CMT2B phenotype in all pedigrees and were not found in 200 control chromosomes. The cumulative LOD score, at 0% recombination, for segregation of the disease-causing mutation in CMT-140 and the small branch of CMT-126 is 8.23, and the LOD score in CMT-90 and CMT-195 is 1.49 and 4.13, respectively. Interestingly, the remaining patients of family CMT-126 (III-1, III-2, III-3, III-6 and IV-6) do not have the Leu129Phe mutation in RAB7. The fact that individuals III-5, IV-2, IV-3 and V-1 of CMT-126 have the same disease-associated haplotype and the same C385T (Leu129Phe) mutation as the patients in CMT-140 (FIG. 1, Panel A), indicates that there is a familial relationship between CMT-140 and a part of CMT-126, who both originate from the South of Austria (Carinthia). The ulcero-mutilating phenotype of the remaining patients in CMT-126 is probably caused by a mutation in another gene (as SPTLC1 is excluded) and further supports the presence of a third locus for ulcero-mutilating neuropathies. The alignment of RAB7 orthologs shows that both missense mutations target highly conserved amino acid residues (FIG. 1, Panel C, SEQ ID NOS:177-184). The Val162Met mutation affects a valine that is conserved among all species. The Leu129Phe mutation is located next to a conserved GTP-binding domain (-NKID-). Leul29 is not conserved in Arabidopsis and yeast. Vitelli et al. reports an expression of two transcripts of 2.5 and 1.8 kb for the human RAB7 gene in different cell types. The expression information of human and mouse RAB7 in the Unigene database suggests ubiquitous expression (Unigene Clusters: Hs.356386 and Mm.4268). Expression was found in all tested tissues. However, in human, the highest level of expression was found in skeletal muscle, while in mouse, the liver, heart and kidney had a high level of expression. Analysis of cDNA from mouse sensory (DRGs) and motor neurons (ventral horn) showed expression of RAB7 in both cell types. In conclusion, two missense mutations in the RAB7 late endosomal protein were reported as the cause for the ulcero-mutilating inherited peripheral neuropathy CMT2B.

2. ARHGEF10

The phenotype of slowed motor and sensory nerve conduction velocities (NCVs) in a four-generation family (CMT-54 family; De Jonghe et al, (1999) Arch. Neurol. 56:1283-1288), was accidentally discovered upon clinical and electrophysiological examination of the proband III-16 for vascular problems of the leg. Subsequent examination identified slowed NCVs in 12 of 39 healthy relatives (5 males, 7 females) indicating an autosomal dominant inheritance of the phenotype. NCVs were uniformly slowed in all nerves examined: 34 to 42 m/s for motor median nerve (normal≧49 m/s), 27 to 36 m/s for motor peroneal nerve (normal≧41 m/s), 32 to 46 m/s for sensory median nerve (normal≧46 m/s), 33 to 45 m/s for sensory ulnar nerve (normal≧46 m/s), and 28 to 35 m/s for sensory sural nerve (normal≧44 m/s). Compound muscle action potentials were normal and sensory nerve action potentials were sometimes slightly reduced. None of the affected family members showed any clinical signs of peripheral or central nervous system dysfunction. The eldest individuals II-4 and II-7, respectively 87 and 78 years old at neurological examination, had NCVs that were not significantly different from those measured in younger affected. Histological studies of a peripheral nerve biopsy of the proband III-16 at 54 years showed numerous relatively thin myelin sheaths (mean g-ratio: 0.75 for myelinated fibers ranging from 2 μm to 7 μm), slight onion bulb formation and few axonal regeneration clusters. Family CMT-54 was excluded of all known loci for inherited peripheral neuropathies, indicating that this family represents a novel clinical and genetic entity of HMSN.

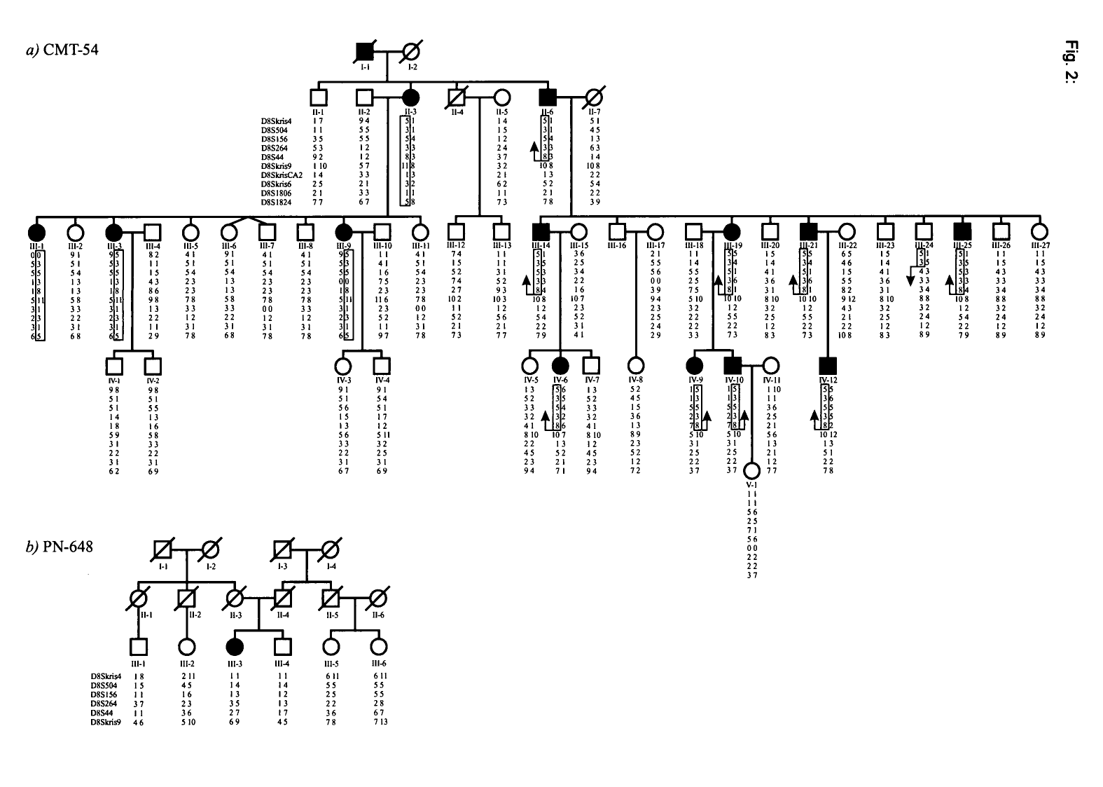

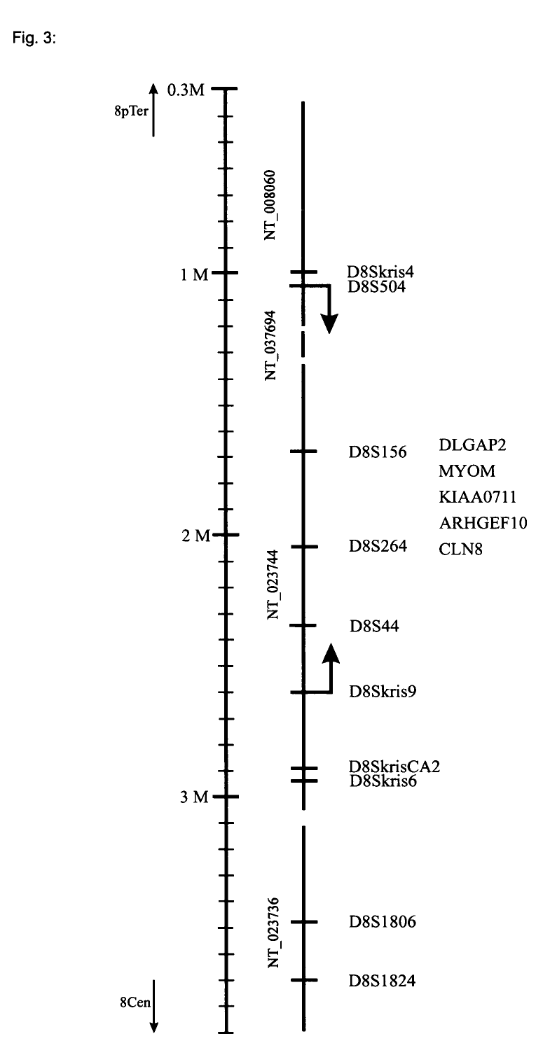

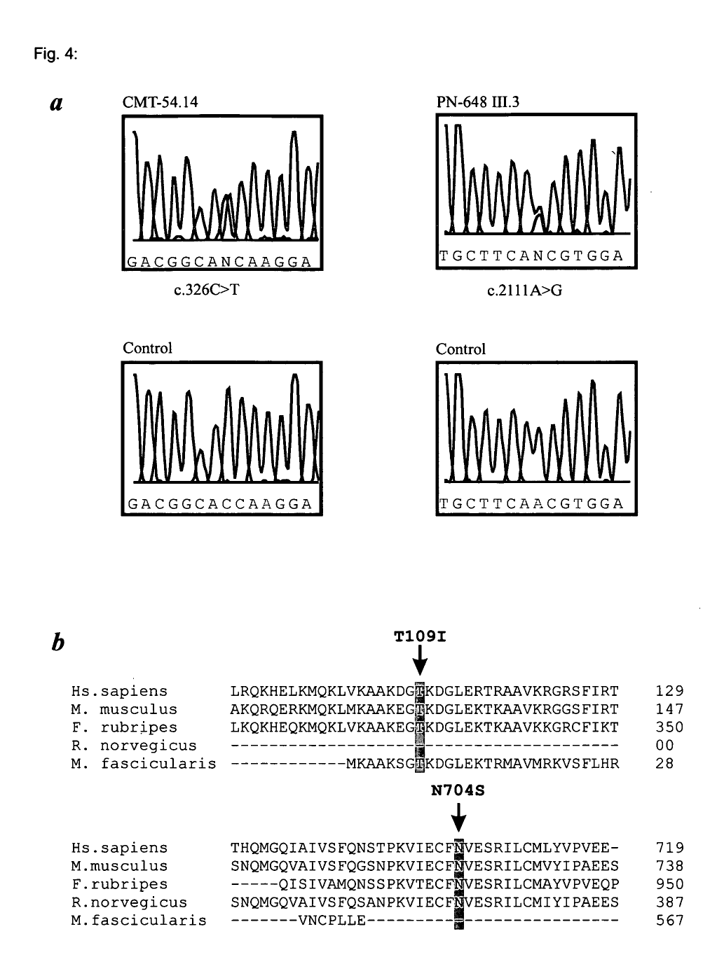

In order to map the disease locus in family CMT-54, a genome-wide scan using 382 short tandem repeat (STR) markers (ABI Prism® Linkage Mapping Set MD-10 (PE Biosystems)), which have an average inter-marker distance of 10 cM, was performed. Significant linkage with STR marker D8S264 on the short arm of chromosome 8 (LOD score=3.01 at 0% recombination, Table 3) was found. To fine-map the disease locus on 8p, five known STR markers (D8S504, D8S44 (AF009213), D8S156 (AF009208), D8S1806 and D8S1824) were selected and four new STR markers (STR1, STR2, STR3 and STR4) flanking D8S264 were identified by using sequence information from public databases (Table 4, FIG. 3). Two-point linkage analysis demonstrated positive LOD scores for all makers tested. A maximum LOD score of 9.33 was obtained with the most informative marker AF009213 (Table 3). For each marker, alleles associated with the HMSN phenotype were identified and a disease haplotype was constructed in family CMT-54 (FIG. 2, Panel A). In patients II-3, III-1, III-3 and III-9, the disease haplotype covers the ten STR markers. Patient II-6 has a recombination with markers STR2, STR3, STR4, D8S1806 and D8S1824, which is inherited by his four affected children (III-14, III-19, III-21 and III-25). The healthy relative III-24 carries a part of the disease haplotype at marker STR1 and D8S504. These recombination events assign the disease locus in family CMT-54 between the telomeric marker D8S504 and centromeric marker STR2. Physical mapping data demonstrated that the region is covered by sequenced clone contigs NT_{—}008060, NT_{—}037694 and NT_{—}023744, representing ±1.5 Mb (NCBI, LocusLink) (FIG. 3). In the novel HMSN locus on chromosome 8p23, five positional candidate genes for mutation analysis were selected: KIAA0711 (hypothetical protein KIAA0711), MYOM2 (myomesin (M-protein] 2), CLN8 (ceroid-lipofuscinosis, neuronal 8), DLGAP2 (discs, large [Drosophila] homolog-associated protein 2) and ARHGEF10 (Rho guanine nucleotide exchange factor 10). For each gene, all known exons and intron-exon boundaries in patients from family CMT-54 and healthy controls were selected. No disease-associated mutations were found in MYOM, CLN8, DLGAP3 and KIAA0711. Subsequently, the 8467 bp mRNA sequence of ARHGEF10 (NM_{—}014629) with the sequence of contig NT_{—}023744 were annotated and 22 coding exons were retrieved, spanning a genomic size of 136160 bp (Table 5). In exon 3 of ARHGEF10, a heterozygous C→T transition mutation at nucleotide coding position 326 (C326T, Thr109lle) in patients III-9 and III-19 was found (FIG. 4, Panel A). This Thr109lle missense mutation co-segregated with the disease phenotype in family CMT-54. Subsequently, 95 patients with an HMSN phenotype, previously excluded for mutations in the common CMT genes PMP22, MPZ and connexin 32 (GJB1), were screened. In patient III-3 of an Italian family (PN-648), an A→G transition at coding position 2111 (A2111G, Asn704Ser) in exon 17 of ARHGEF10 was found (FIG. 4, Panel A). Since the parents of patient III-3 were not available for mutation analysis, no determination could be made as to whether the Asn704Ser mutation occurred de novo or whether it was inherited as an autosomal dominant trait. The patient's healthy brother and other relatives did not carry the Asn704Ser mutation or the disease-associated haplotype with STR markers from the novel HMSN locus on chromosome 8p23 (FIG. 2, Panel B, SEQ ID NOS:189-197). Both missense mutations, Thr109lle in CMT-54 and Asn704Ser in PN-648, were not found in 600 normal control chromosomes. The ARHGEF10 protein contains 1121 amino acids and contains a conserved dbl homology (DH) domain from codon 177 to 359 (ScanProsite, http://us.expasy.org/cgi-bin/scanprosite).

The CLUSTALW protein alignment of human ARHGEF10, macaque, puffer fish, rat and mouse Gef10 orthologues showed that the Thr109lle and Asn704Ser missense mutations target highly conserved amino acid residues (FIG. 4, Panel B, SEQ ID NOS:189-197).

Expression of ARHGEF10 using its mouse orthologue Gef10 was examined. Alignment of the Gef10 transcript of 4481 bp (NM_{—}172751) with the genomic sequence NT_{—}039455 identified 24 exons, exons 1 and 2 being absent from ARHGEF10. Multiple tissue Northern blot analysis of Gef10 indicated ubiquitous expression. Overlapping primer sets covering the mouse cDNA sequence were used in PCR analysis on cDNA of E13 mouse brain, dorsal root ganglia (DRG) and ventral horn (VH) and demonstrated Gef10 expression in all three neuronal tissues. Extra PCR fragments were observed that indicated the presence of alternative transcripts. Sequencing of these fragments identified three splice variants of Gef10: one in all three tissues missing exon 4, one specific for DRG missing exon 21, and one specific for VH with an insertion of an additional exon of 165 bp between exons 22 and 23. Exon 5 corresponding with exon 3 in ARHGEF10 and containing the Thr109lle mutation, is present in all three variant transcripts. Whole mount in situ hybridization experiments in mouse embryos at E8.5 showed Gef10 expression in the neuroepithelium of the meninges, including the optic sulcus. At E9.5, high levels of Gef10 expression were detected in the roof plate of the rhombencephalon. In E12.5 embryos, Gef10 is ubiquitously expressed with a pronounced expression in the neuroepithelium of brain vesicles, the neural tube, the ganglia, DRG and the neural layer of the retina.

ARHGEF10 encodes a guanine nucleotide exchange factor for the Rho family of GTPase proteins (RhoGEFs), and contains a DbI homology (OH) domain (codons 177 to 359), a common feature of all RhoGEFs. RhoGEFs activate RhoGTPases by catalyzing the exchange of bound GDP for GTP, inducing a conformational change in the GTP-bound GTPase that allows its interaction with downstream effector proteins. Within the RhoGEF family, DH domains are invariably followed by a pleckstrin homology (PH) domain supposed to be involved in subcellular localization of RhoGEFs. However, in ARHGEF10, a PH domain consensus motif using several bioinformatic tools (BlastP, ScanProsite or InterPro) was not detected. So far, only one other mammalian RhoGEF family member, p164-RhoGEF, lacking the PH domain has been reported. ARHGEF10 appears to lack an equivalent protein in C. elegans, D. melanogaster, D. discoideum and S. cerevisiae, suggesting that the ARHGEF10 signaling pathway is unique to vertebrates. This confirms the overall picture of plasticity when comparing the RhoGTPases and their interacting proteins between species, with certain species gaining or losing RhoGTPase and RhoGEF family members to give rise to unique sets of signaling proteins. RhoGTPases play a pivotal role in regulating the actin cytoskeleton but their ability to influence cell polarity, microtubule dynamics, membrane transport pathways and transcription factor activity is probably just as significant. Recent evidence has implicated RhoGTPases in neuronal morphogenesis, including cell migration, axonal growth and guidance, dendrite elaboration and plasticity, and synapse formation. Several GEFs play a central role in defining the temporal and spatial activation of the corresponding GTPase within neuronal cells.

The identification of ARHGEF10 as a gene implicated in peripheral nerve conduction raises questions about its role during the development of the peripheral nervous system in vertebrates. All affected members in the family had slowed NCVs with normal amplitudes at all ages, indicating that the phenotype is non-progressive. Together with the numerous thin myelinated axons in the absence of gross signs of demyelination or axonal de- and regeneration in the peripheral nerve biopsy of the proband, without wishing to be bound by theory, one possible theory is that these observations are indicative of a congenital non-progressive phenotype, suggesting that ARHGEF10 is most likely involved in normal development of peripheral nerves.

### Materials and Methods

1. RAB7

1.1. Family Material

The study described herein comprised three families previously linked to the CMT2B locus on 3q13-q22, the originally described American CMT2B family,^{7, 20} a Scottish family CMT-90,⁸ and an Austrian family CMT-140.⁹ In addition, another Austrian family, CMT-126, previously excluded for the CMT2B and HSN I loci,³ was studied. In summary, the clinical picture of CMT2B is mild to severe, with sensory loss and all modalities equally affected. Spontaneous pain is absent. Motor deficits are often the first and most prominent sign of the disease. The distal leg muscles are more affected than the hand muscles. Nerve conduction velocity (NCV) studies indicate an axonal neuropathy that allows clinical diagnosis in asymptomatic individuals (reviewed in reference 2). Genomic DNA from total blood samples from family members and control persons using a standard extraction protocol was isolated. Informed consent was obtained from all family members and this study was approved by the Institutional Review Board at the Universities of Antwerp, Edinburgh, Graz, and St Louis.

1.2. Molecular Genetics

From sequences of human High Throughput Genomic Sequences (HTGS) clones localized in the CMT2B region (NT_{—}031776, NT_{—}005543, NT_{—}005588, NT 028133, NT_{—}022513, NT_{—}005523, NT_{—}006025, NT_{—}022404, NT 005823), known STR sequences were selected and six new STR markers by BLASTN searches were identified (NCBI site at http://www.ncbi.nlm.nih.gov/BLAST/): D3SCMT126A, D3SCMT126B, D3SCMT126C, D3SCMT126D, D3SCMT126F and D3SCMT126G. For genotyping STRs, primer pairs were designed. PCR amplification was performed with dye-labeled primers on a DYAD thermocycler (MJ Research). Fragment analysis was performed on an ABI3700 DNA sequencer and analyzed with the ABI GENESCAN 3.1 and GENOTYPER 2.1 software (Perkin-Elmer, Applied Biosystems Inc.). Genetic linkage was computed with the LINKAGE program (http://linkage.rockefeller.edu/) considering the disease-causing mutation as rare allele (1%), equal male and female recombination fractions and a disease frequency of 1/10,000.

1.3. Mutation Analysis

The NCBI Entrez Genome Map Viewer (http://www.ncbi.nlm.nih.gov/cgi-bin/Entrez/hum_srch?chr=hum_chr.inf&query), Ensembl Human Genome Server (http://www.ensembl.org/) and Genbank database (http://www.ncbi.nlm.nih.gov/entrez/query.fcgi?db=Nucleotide) were used to find known genes, ESTs and putative novel genes in the CMT2B region. The exon-intron boundaries of the candidate sequences were determined by BLAST searches against the HTGS. All exons of the ZNF9, ABTB1 and RAB7 genes were PCR-amplified using intronic primers (Table 2, SEQ ID NOS:3-70). PCR products were sequenced using the DYEnamic ET Terminator Cycle Sequencing Kit (Amersham Pharmacia Biotech). The sequence reactions were loaded on the ABI3700 sequencer (Perkin-Elmer, Applied Biosystems Inc.). The data were collected and analyzed using the ABI DNA sequencing analysis software, version 3.6.

1.4. Expression Analysis

Three plasmid clones, IMAGp956B0837, IMAGp956M0263 and IMAGp956M2246, containing partial human RAB7 cDNA sequences were obtained from RZPD (The Resource Center of the German Human Genome Project at http://www.rzpd.de/). T3- and T7-primers were used to make a RAB7 cDNA probe of 800 bp. This probe was used to hybridize the Human 12-lane Multiple Tissue Northern blot (Clontech).

Total RNA was extracted from mouse brain (NMRI) using the Totally RNA Kit (Ambion). RT-PCR was carried out using the SMART RACE cDNA Amplification kit (Clontech). The full length mouse RAB7 cDNA was cloned into the pCRII-TOPO vector (Invitrogen) and used as a probe to hybridize the Mouse Multiple Tissue Northern blot (Clontech). Both Northern blots were also hybridized with a β-actin cDNA probe (Clontech) as a control for RNA loading.

Motor and sensory neurons were isolated from 13 day-old mice embryos. Total RNA was extracted using the Totally RNA Kit (Ambion) and RT-PCR was carried out using the SMART RACE cDNA Amplification kit (Clontech). Mouse RAB7 cDNA primers (MRAB7-2F=5′-CTGACCAAGGAGGTGATGGT-3′ (SEQ ID NO:1) and MRAB7-2R=5′-GAACAGTTCTCACTCTCC-3′ (SEQ ID NO:2)) were used to amplify a RAB7 cDNA fragment of 854 bp.

1.5. Genbank Accession Numbers

Protein sequences: RAB7_human, P51149 (SEQ ID NO:170); RAB7_mouse, P51150 (SEQ ID NO:178); RAB7 rat, P09527 (SEQ ID NO:179); Rab-protein 7 Drosophila melanogaster, NP_{—}524472 (SEQ ID NO:180); RAB7_{—}Dictyostelium discoideum, P36411 (SEQ ID NO:181); Ras-related protein_{—}Caenorhabditis elegans, NP 496549 (SEQ ID NO:182); RAB7_{—}Arabidopsis thaliana, O04157 (SEQ ID NO:183); YPT7_YEAST, P32939 (SEQ ID NO:184).

2. ARHGEF10

2.1 Electronic Database Information

Accession numbers and URLs for data presented herein are as follows:

ClustalW, http://npsa-pbil.ibcp.fr/ (for multiple protein alignment).

GenBank, http://www.ncbi.nlm.nih.gov/entrez/query.fcgi?db=Nucleotide for mRNA sequences: Myomesin (M-protein) 2 (MYOM2), NM_{—}003970; KIAA0711, NM_{—}014867; Rho guanine nucleotide exchange factor (GEF) 10 (ARHGEF10), NM_{—}014629; ceroid-lipofuscinosis neuronal 8 (CLN8), NM_{—}018941; discs, large (Drosophila) homolog-associated protein 2 (DLGAP2), NM_{—}004745. For Protein sequences: Homo sapiens Rho guanine nucleotide exchange factor (GEF) 10, NP_{—}055444; Mus musculus sequence similar to GEF10, NP-766339; Macaca fascicularis brain cDNA similar to GEF10, BAB12119; Rattus norvegicus protein similar to GEF10, XP_{—}225032; Fugu rubripes.

NCBI Map Viewer, (http://www.ncbi.nlm.nih.gov/cgi-bin/Entrez/hum_srch?chr=hum_chr.inf&guery (for finding known genes, ESTs, and putative novel genes in the 8p23 region.

Online Mendelian Inheritance in Man (OMIM), http://www.ncbi.nlm.nih.gov/Omim/ (for CMT (MIM # 118220)).

### TABLES

TABLE 1A
Mutations in RAB7 causing CMT2B

| | | Mutation |
|---|---|---|
| Exon | Family/Patient | (cDNA, Protein) |
| 3 | CMT-140, CMT-126 (part), CMT-1 86.26 | C385T, Leu129Phe |
| 4 | CMT-90, CMT-195, CMT-186.28, PN-626.1 | G484A, Val162Met |

TABLE 1B
Mutations in ARHGEF10 causing HMSN

| Exon | Family/Patient | Mutation (cDNA, Protein) |
|---|---|---|
| 3 | CMT-54 | C326T, Thr109lle |
| 17 | PN-648 | A2111G, Asn704Ser |

TABLE 2

The exons of the ZNF9, ABTB1 and RAB7 genes were PCR-amplified using the following intronic primers
(SEQ ID NOS:3-70, in sequential order, left to right and top to bottom):

| | | | | | | PCR |
|---|---|---|---|---|---|---|
| Gene | Exon | Forward primer | 5-intron/exon | 3′-intron/exon | Reverse primer | length |
| ZNF9 | exon | 5′-GATTACTGGCGTGAGCCACC-3′ | cagccATGAG | TAGAGgtatt | 5′-GGCTAGTCAGACCAGTCTTG-3′ | 361 bp |
| | 1 | | | | | |
| | exon | 5′-CAAGACTGGTCTGACTAGCC-3′ | aacagGTTTC | GGATGgtaag | 5′-CACAGTTGCATGTGCTCACC-3′ | 291 bp |
| | 2 | | | | | |
| | exon | 5′-GGTGAGCACATGCAACTGTG-3′ | tgaagCCTGC | TATAGgtaag | 5′-GCCACAGGATATCAGGGCAG-3′ | 511 bp |
| | 3 | | | | | |
| | exon | 5′-GCATGGTGACTGTTGCTTTG-3′ | tttagGTGTG | CCTAAttatt | 5′-GGGAGTTGCCTCTATCTGCC-3′ | 290 bp |
| | 4 | | | | | |
| ABTB1 | exon | 5′-GTCTGATGAGCCTGGCCCAG-3′ | agaccATGGG | TGCAGgtagc | 5′-GGCAAGGAAATGGTCACCTG-3′ | 331 bp |
| | 1 | | | | | |
| | exon | 5′-CATGACCTGATGACCCCTGC-3′ | cttagATTAT | | 5′-GAAAAGTCACGGACCTGAGG-3′ | 394 bp |
| | 2A | | | | | |
| | exon | 5′-CTGGCATATGCCTCCCAGAG-3′ | | | 5′-CGCTTGCTTCCTGAGGAGCC-3′ | 395 bp |
| | 2B | | | | | |
| | exon | 5′-CTCCACCAGCCAAGAGGCAG-3′ | | CGCAGgtaaa | 5′-GCCAGGCTCTGTGAAAGAAG-3′ | 410 bp |
| | 2C | | | | | |
| | exon | 5′-CACTGGCCTCCACCTTCCAG-3′ | cacagGTGAT | ACTGTgtgag | 5′-CACTGCCTGCAGCCTTTCCC-3′ | 234 bp |
| | 3 | | | | | |
| | exon | 5′-GAGGGTTGAGGGTGAGTACG-3′ | tgtagGAGGT | GCAGGgtgag | 5′-CAAAGCCCTTATGCGTGCAC-3′ | 351 bp |
| | 4 | | | | | |
| | exon | 5′-GAGACAGAGGCTCAGGCCAG-3′ | cccagGGGTG | AGGAGgtaag | 5′-CGATGTGGGCAGACTCCTGG-3′ | 254 bp |
| | 5 | | | | | |
| | exon | 5′-CCAATGCAACCACCCCAGAG-3′ | cctagATTGG | ACGTGgtgag | 5′-GCAGATGAGGACACCTATCG-3′ | 326 bp |
| | 6 | | | | | |
| | exon | 5′-GATCGATGACGTCAGGGAGG-3′ | ctcagTCGCA | TGTGAgcgca | 5′-CCGAGGCCGATCCAGTTATC-3′ | 510 bp |
| | 7 | | | | | |
| RAP7 | exon | 5′-GGCTGCTCAGACATTTGTGC-3′ | gaaggATGAC | TCTGGgtaag | 5′-GAAGTGGCAGCACGGACAGT-3′ | 233 bp |
| | 1 | | | | | |
| | exon | 5′-GTCCTTCAGGTCAGGCAGATT-3′ | ttcagAGTCG | TGCAGgtaag | 5′-CTGAGTATCAGCCAATTATC-3′ | 365 bp |
| | 2 | | | | | |
| | exon | 5′-GCACCCCTTGCATACATGCT-3′ | cacagATATG | GACAAgtaag | 5′-GTGAGCTTAGCAGAGAACC-3′ | 377 bp |
| | 3 | | | | | |
| | exon | 5′-GAGGATGGAGTCAGTGCTGG-3′ | ttcagGTGGC | AGCAGgtggg | 5′-GTCAGTGGTCAGGCATCACC-3′ | 244 bp |
| | 4 | | | | | |
| | exon | 5′-CACTCTGCCCAAGCAGAAGTG-3′ | tccagGAAAC | CTGAgggggc | 5′-GGAAGAGGAGAGGGGAATTG-3′ | 288 bp |
| | 5 | | | | | |

TABLE 3
Two-point linkage results with chromosome 8p23 markers.
LOD scores at recombination fraction Θ

| | 0 | 0.001 | 0.01 | 0.05 | 0.1 | 0.2 | 0.3 | 0.4 |
|---|---|---|---|---|---|---|---|---|
| STR1 | 6.32 | 6.31 | 6.22 | 5.81 | 5.26 | 4.05 | 2.68 | 1.19 |
| D8S504 | −∞ | 6.20 | 7.07 | 7.20 | 6.76 | 5.44 | 3.79 | 1.87 |
| AF009208 | 6.98 | 6.97 | 6.87 | 6.41 | 5.82 | 4.58 | 3.19 | 1.60 |
| D8S264 | 3.01 | 3.01 | 2.96 | 2.77 | 2.51 | 1.95 | 1.34 | 0.68 |
| Thr109lle | 9.93 | 9.91 | 9.78 | 9.17 | 8.37 | 6.64 | 4.68 | 2.46 |
| AF009213 | 9.33 | 9.32 | 9.19 | 8.60 | 7.82 | 6.14 | 4.24 | 2.09 |
| STR2 | −∞ | 4.39 | 5.28 | 5.51 | 5.19 | 4.15 | 2.84 | 1.34 |
| STR3 | −∞ | 4.27 | 5.18 | 5.44 | 5.18 | 4.25 | 3.02 | 1.53 |
| STR4 | −∞ | 5.36 | 6.25 | 6.43 | 6.05 | 4.90 | 3.44 | 1.72 |

Legend: Two-point linkage analysis was performed using the MLINK program of the FASTLINK program package (Cottingham R. W. et al. (1993) Am. J. Hum. Genet. 53:252-263; Lathrop G. M. and Lalouel J.-M. (1984) Am. J. Hum. Genet. 36:460-465). Since NCV values were diagnostic in all individuals, the phenotype was coded as a 100% penetrant phenotype (De Jonghe et al. (1999) Arch. Neurol. 56:1283-1288). The gene frequency was set at 0.0001, allele frequencies were set at 1/N (N=number of alleles observed in the pedigree), and equal recombination rates between males and females were assumed. Sequences of the four new STR markers, STR1, STR2, STR3 and STR4, can be found in Table 4.

TABLE 4
Primer conditions for new STR markers on chromosome 8p23 (SEQ ID NOS:71-78):

| | | | | | |
|---|---|---|---|---|---|
| STR | Primer pair: | PCR product | # | NT | Position in NT |
| marker | F = forward, R = reverse | length | alleles | contig | sequence |

| | | | | | |
|---|---|---|---|---|---|
| STR1 | F: 5′-CCTCATTCTGCAGCGAGATGG-3′ | 285-307 bp | 11 | NT_008060 | 656446-656730 |
| | R: 5′-TGGACAGAGGCATGAGGAAGAC-3′ | | | | |
| STR2 | F: 5′-GTGCAGATTCACTGCTGCTAAC-3′ | 188-216 bp | 13 | NT_023744 | 1244480-1244663 |
| | R: 5′-GAGCGAGAGAGACCACTGTAT-3′ | | | | |
| STR3 | F: 5′-GTTTGCTCATCTTGTACAGTGC-3′ | 169-177 bp | 4 | NT_023744 | 1522783-1522955 |
| | R: 5′-CTGCAGTCCACTCTGGAAACA-3′ | | | | |
| STR4 | F: 5′-CCAAAAACCTTCAGCTGAGTC-3′ | 139-159 bp | 6 | NT_023744 | 1589656-1589798 |
| | R: 5′-CAGGACGATATACGTGCACAC-3′ | | | | |

TABLE 5

Oligonucleotide primer sequences used for mutation analysis and intron-exon boundaries of ARHGEF10
(SEQ ID NOS:79-168, in sequential order, left to right, top to bottom).

| | | | | | | | |
|---|---|---|---|---|---|---|---|
| | | | | | | PCR | |
| Ex- | Size | | 5′-intron/ | 3′-exon/ | | product | IVS |
| on | (bp) | Forward primer | exon | intron | Reverse primer | (bp) | (bp) |

| | | | | | | | |
|---|---|---|---|---|---|---|---|
| 1 | 174 | 5′-CCCCAGCTCTAGATGATTTGG-3′ | aattt/ATGCA | AGCAA/gtacg | 5′-GGCAAAGGAGAAGACGTGTC-3′ | 424 | 3313 |
| 2 | 117 | 5′-CTGCCAGCATCCTCTCAATG-3′ | tgtag/CTTTC | TGAAG/gtaga | 5′-GTCCTTGCTGTCTCAACAGC-3′ | 294 | 2472 |
| 3 | 115 | 5′-CGTGACACATGCGCTCAGAAG-3′ | cgcag/ATGCA | ACAGG/gtccg | 5′-CTGCTCAGTGCTTGGGTCTG-3′ | 468 | 2851 |
| 4 | 107 | 5′-GCTGGGTGTCAAGTAAGCATG-3′ | ttaag/ATCAC | AGCAG/gtgag | 5′-GCTGTCTCAAACTCTGCATCC-3′ | 353 | 7863 |
| 5 | 78 | 5′-CAGCAAGCCTCAGCATAGAAG-3′ | aacag/GTTGT | TGGAG/gtact | 5′-GGATCTAAGTATTATGTCTG-3′ | 312 | 747 |
| 6 | 180 | 5′-GCTGCAGTGAGCCATGATCG-3′ | cttag/CAATA | CTTCG/gtaat | 5′-CTGATCTCCTGCAAGCTGAC-3′ | 390 | 1760 |
| 7 | 117 | 5′-GATCACAGTGACCGAAAGAG-3′ | ttcag/TTTTC | TAAAG/gtaag | 5′-CAGTATCAATAGTGCCCTAG-3′ | 349 | 1983 |
| 8 | 93 | 5′-GAGTGTTCAGTGTGGTGGGG-3′ | cacag/CAGGA | TCCAG/gtaag | 5′-GTTCCTCCACTTTGGAATGGC-3′ | 277 | 4756 |
| 9 | 171 | 5′-CTCCTTTCCATTGTCAGCTG-3′ | tgtag/GACAT | ACAAG/gttga | 5′-CTGTCGTTGTCCAGCAATAC-3′ | 342 | 2119 |
| 10 | 146 | 5′-CCTGAGACTCCATACCAGAC-3′ | tccag/CTTCT | TGAGG/gtaag | 5′-CTGTCACTGAGACTGAACTGG-3′ | 338 | 3578 |
| 11 | 176 | 5′-CAGCAACGGGAAGTTTCTCAC-3′ | ttcag/GCCCT | ACCAA/gtaag | 5′-GTAAGCTCCACCATCAGCAG-3′ | 372 | 13675 |
| 12 | 116 | 5′-GTTCTAGATTCACCCCTCAAC-3′ | tgtag/ACAAA | ATCAG/gtaac | 5′-CCAAGTTCTACCAGAAGTGAG-3′ | 331 | 388 |
| 13 | 128 | 5′-CAGTGTGATCTGACTCCCAAAG-3′ | tgtag/AACTT | GACAA/gttag | 5′-TGCTGATTCTATCAGACAGGC-3′ | 387 | 178 |
| 14 | 101 | 5′-GCTGATTCTATCAGACAGGC-3′ | ggcag/ATCTG | CCTAG/gtaag | 5′-GTGAGGTCAGGGTTGAGAAG-3′ | 278 | 1408 |
| 15 | 122 | 5′-CAGAATGCCAGAAACTTCCCC-3′ | tgcag/AAGAG | TCAAG/gtgaa | 5′-GTTCACCACAGTGACCGCTTC-3′ | 353 | 972 |
| 16 | 87 | 5′-GTAGTCTAGGAGCCTCTTAGC-3′ | tttag/ATTGA | TGTGG/gtaag | 5′-TTGCTCAGTAGAGAGTTGGCG-3′ | 252 | 1963 |
| 17 | 224 | 5′-GAAAATCGAAGCTTGTCGTGGC-3′ | ttcag/ATCGG | GGAAG/gtagg | 5′-GAGTCCTTGACTTTGACTCAGG-3′ | 425 | 635 |
| 18 | 158 | 5′-GCAAGTGTCCCTAGGAATGG-3′ | taaag/CATTT | CCCAG/gtgag | 5′-GACTGTGCATCCGGTTTAGC-3′ | 360 | 4359 |
| 19 | 143 | 5′-CATTACAGGTGATCCTTCGGG-3′ | ttaag/ATGGA | TAGAG/gtaag | 5′-GTGCCGAAGTCAAGGCTTC-3′ | 341 | 11574 |
| 20 | 175 | 5′-CTACTGTGTTGCAGCCAGTG-3′ | cacag/GGTCA | GCCAG/gtaag | 5′-CTTAGCGCTTCGCAGTGCAG-3′ | 386 | 7098 |
| 21 | 123 | 5′-GATTTGGTGGTGGCACGACA-3′ | cccag/GGCAC | GACCG/gtgag | 5′-CTCGTGGAGCATAGCAGTG-3′ | 295 | 3936 |
| 22 | 515 | 5′-GGAATGCGTTGGGGTTAAGC-3′ | ttcag/GAAGA | | 5′-CTGAGCTTGTCTCACGGCTC-3′ | 380 | |
| A | | | | | | | |
| 22 | | 5′-GAGTGGAGGAGCTGGTTCATC-3′ | | TATAA/gcagg | 5′-GCTGTGTCTACACTGGTTGG-3′ | 462 | |
| B | | | | | | | |

Seq ID (SEQ ID NOS:169-172, in sequential order)

SEQ ID NO:169
atgacctcta ggaagaaagt gttgctgaag gttatcatcc tgggagattc tggagtcggg
aagacatcac tcatgaacca gtatgtgaat aagaaattca gcaatcagta caaagccaca
ataggagctg actttctgac caaggaggtg atggtggatg acaggctagt cacaatgcag
atatgggaca cagcaggaca ggaacggttc cagtctctcg gtgtggcctt ctacagaggt
gcagactgct gcgttctggt atttgatgtg actgccccca acacattcaa aaccctagat
agctggagag atgagtttct catccaggcc agtccccgag atcctgaaaa cttcccattt
gttgtgttgg gaaacaagat tgacctcgaa aacagacaag tggccacaaa gcgggcacag
gcctggtgct acagcaaaaa caacattccc tactttgaga ccagtgccaa ggaggccatc
aacgtggagc aggcgttcca gacgattgca cggaatgcac ttaagcagga aacggaggtg
gagctgtaca acgaatttcc tgaacctatc aaactggaca agaatgaccg ggccaaggcc
tcggcagaaa gctgcagttg ctga
SEQ ID NO:170
MTSRKKVLLKVIILGDSGVGKTSLMNQYVNKKFSNQYKATIGADFLTKEVMVDDRLVTMQIWDTAGQ
ERFQSLGVAFYRGADCCVLVFDVTAPNTFKTLDSWRDEFLIQASPRDPENFPFVVLGNKIDLENRQV
ATKRAQAWCYSKNNIPYFETSAKEAIVEQAFQTIARNALKQETEVELYNEFPEPIKLDKNDRAKASA
ESCSC
SEQ ID NO:171
atgcactcag atgaaatgat ttatgatgat gttgagaatg gggatgaagg tggaaacagc
tccttggaat acggatggag ttcgagtgaa tttgaaagtt acgaagagca gagtgactcg
gagtgcaaga atgggattcc caggtccttc ctgcgcagca accacaaaaa gcaactttct
catgacctaa cccgtttaaa ggagcactat gagaaaaaga tgagagattt gatggcaagc
acggtgggcg tggtggagat tcagcagctc aggcagaagc atgaactgaa gatgcagaag
ctcgtgaagg ccgcgaagga cggcaccaag gacgggctgg agaggaccag ggcagccgtg
aagaggggcc gctccttcat caggaccaag tctctcatcg cacaggatca cagatcttct
cttgaggaag aacagaattt gttcattgat gttgactgca agcacccgga agccatcttg
accccgatgc ccgagggttt atctcagcag caggttgtaa gaagatatat actgggttca
gttgtcgaca gtgaaaagaa ctacgtagat gctcttaaga ggattttgga gcaatatgag
aagccgctgt ctgagatgga gccaaaggtt ctgagtgaga ggaagctgaa gacggtgttc
taccgagtca aagagatcct gcagtgccac tcgctatttc agatcgcgct ggccagccgc
gtttccgagt gggactccgt ggaaatgata ggcgatgtct tcgtggcttc gttttctaag
tccatggtgc tggatgcata cagtgaatat gtgaacaatt tcagcacagc cgtggcagtc
ctcaagaaaa catgtgccac aaagcccgct tttcttgaat ttttaaagca ggaacaggag
gccagccccg atcgaaccac gctctacagc ctgatgatga agcccatcca gaggttccca
cagttcatcc tcctgctcca ggacatgctg aagaacacct ccaaaggcca ccccgacagg
ctgcctcttc agatggccct gacagagctc gaaacactag cagagaagtt aaatgaaaga
aagagagatg ctqatcaacg ctgtgaagtg aagcaaatag ccaaagccat aaacgaaaga
tacctgaaca agcttctcag cagtggaagc cgatacctca ttcgatcaga tgatatgata
gaaacagttt acaacgacag aggagagatt gttaaaacca aagaacgccg agtcttcatg
ttaaatgatg tgttaatgtg tgccaccgtc agctcacgcc cctctcatga cagccgtgtg
atgagcagcc agaggtactt gctgaagtgg agcgttccac tgggacatgt ggacgccatc
gagtatggca gcagcgcagg cacgggcgag cacagcaggc accttgccgt tcacccgccg
gagagcctgg ccgtggttgc taacgcgaaa ccaaacaaag tttacatggg gccaggacaa
ctgtatcaag atttacaaaa cttgttgcat gacttaaatg taattggcca aatcactcag
ctgataggaa accttaaagg aaactatcag aacttaaacc agtcagtagc ccatgactgg
acatcaggtt tacaaaggct tattttgaag aaagaagatg aaatcagagc tgcggactgc
tgcagaattc agttacagct tcccgggaag caggacaaat ctgggcgacc gacgttcttt
acagctgtgt tcaatacgtt cacccctgcc atcaaggagt cctgggtcaa cagcttacag
atggccaagc tcgccctaga agaggagaac cacatgggct ggttctgtgt ggaagacgat
gggaatcaca ttaaaaagga gaagcatcct ctcctcgtcg gacacatgcc cgtgatggtg
gccaagcagc aggagttcaa gattgaatgt gctgcttata accctgaacc ttacctaaat
aatgaaagcc agccagattc attttccacg gcacatggtt tcctgtggat cggaagttgc
acccatcaaa tgggtcagat tgccatcgtc tcgtttcaaa attccactcc caaagtcatt
gagtgcttca acgtggaatc tcgcatcctg tgcatgctgt acgttcccgt cgaggagaag
cgcagagagc ctggggcacc cccggacccc gagaccccgg ccgtgagagc ttctgatgtc
cccacgatct gtgtagggac ggaggaggga agcatttcca tttataaaag cagtcaaggc
tccaagaaag tgagacttca gcactttttc actcctgaga agtccacagt catgagcctg
gcttgcacgt ctcagagcct gtacgctggc ctggtcaacg gggcagtcgc cagctacgcc
agagccccag atggatcctg ggattcagaa cctcaaaaag tgatcaagtt aggcgtccta
ccagttagaa gtctactcat gatggaagac acgttgtggg cggcttccgg aggtcaagtc
ttcatcatca gtgtggagac tcatgctgta gagggtcagc tggaggccca ccaggaggaa
ggcatggtga tctcccacat ggccgtgtcc ggcgtcggga tctggattgc cttcacctca
gggtccacgc tccgcctttt tcacacggaa actctcaagc acctgcagga catcaacatc
gccacccctg ttcacaacat gctgccaggg caccagcggc tgtcggtgac gagcctgctc
gtctgccacg gattgctgat ggtcggcacc agcctgggag tcctcgtggc cctgccggtc
ccacgtctgc aagggattcc caaagtgacc ggaagaggca tggtctccta ccatgcacac
aacagtcctg tcaaattcat cgtcctggcc acggctctgc acgagaaaga caaggacaaa
tccagggaca gcctggctcc tggccccgag cctcaggacg aagaccagaa ggacgcactt
ccgagtggag gagctggttc atctctgagc cagggtgacc ctgacgcagc catctggttg
ggagattcgc tgggatcgat gactcagaaa agcgacctgt cctcctcatc tgggtccctg
agcttgtctc acggctccag ctctctagag cacagatcag aggacagcac catctatgat
ctcctgaagg atcctgtctc gctgagaagc aaagcacgcc gggccaagaa agccaaggcc
agctcggcgc tggtggtctg tggagggcag ggccaccgcc gggtgcacag gaaggcccgg
cagccccacc aggaagagct ggcgccgacc gtcatggtct ggcagatccc tctgctgaat
atataa
SEQ ID NO:172
MHSDEMIYDDVENGDEGGNSSLEYGWSSSEFESYEEQSDSECKNGIPRSFLRSNHKKQLSHD
LTRLKEHYEKKMRDLMASTVGVVEIQQLRQKHELKMQKLVKAAKDGTKDGLERTRAAVK
RGRSFIRTKSLIAQDHRSSLEEEQNLFIDVDCKHPEAILTPMPEGLSQQQVVRRYILGSVVDSE
KNYVDALKRILEQYEKPLSEMEPKVLSERKLKTVFYRVKEILQCHSLFQIALASRVSEWDSV
EMIGDVFVASFSKSMVLDAYSEYVNNFSTAVAVLKKTCATKPAFLEFLKQEQEASPDRTTLY
SLMMKPIQRFPQFILLLQDMLKNTSKGHPDRLPLQMALTELETLAEKLNERKRDADQRCEV
KQIAKAINERYLNKLLSSGSRYLIRSDDMIETVYNDRGEIVKTKERRVFMLNDVLMCATVSS
RPSHDSRVMSSQRYLLKWSVPLGHVDAIEYGSSAGTGEHSRHLAVHPPESLAVVANAKPNK
VYMGPGQLYQDLQNLLHDLNVIGQITQLIGNLKGNYQNLNQSVAHDWTSGLQRLILKKEDE
IRAADCCRIQLQLPGKQDKSGRPTFFTAVFNTFTPAIKESWVNSLQMAKLALEEENHMGWFC
VEDDGNHIKKEKHPLLVGHMPVMVAKQQEFKIECAAYNPEPYLNNESQPDSFSTAHGFLWI
GSCTHQMGQIAIVSFQNSTPKVIECFNVESRILCMLYVPVEEKRREPGAPPDPETPAVRASDV
PTICVGTEEGSISIYKSSQGSKKVRLQHFFTPEKSTVMSLACTSQSLYAGLVNGAVASYARAP
DGSWDSEPQKVIKLGVLPVRSLLMMEDTLWAASGGQVFIISVETHAVEGQLEAHQEEGMVI
SHMAVSGVGIWIAFTSGSTLRLFHTETLKHLQDINIATPVHNMLPGHQRLSVTSLLVCHGLL
MVGTSLGVLVALPVPRLQGIPKVTGRGMVSYHAHNSPVKFIVLATALHEKDKDKSRDSLAP
GPEPQDEDQKDALPSGGAGSSLSQGDPDAAIWLGDSLGSMTQKSDLSSSSGSLSLSHGSSSLE
HRSEDSTIYDLLKDPVSLRSKARRAKKAKASSALVVCGGQGHRRVHRKARQPHQEELAPTV
MVWQIPLLNI

### REFERENCES

1. De Jonghe P., Timmerman V., and Nelis E. Hereditary Peripheral Neuropathies in Neuromuscular Diseases: From Basic Mechanisms to Clinical Management 128-146. Karger, Base! (2000).
2. Auer-Grumbach M. at al. Autosomal dominant inherited neuropathies with prominent sensory loss and mutilations: a review. Arch. Neurol. (2003) 60(3):329-34.
3. Auer-Grumbach M., Wagner K., Timmerman V., De Jonghe P. and Hartung H. P. Ulcero-mutilating neuropathy in an Austrian kinship without linkage to hereditary motor and sensory neuropathy IIB and hereditary sensory neuropathy I loci. Neurology 54, 45-52 (2000).
4. Bellone E. et al. A family with autosomal dominant mutilating neuropathy not linked to either Charcot-Marie-Tooth disease type 2B (CMT2B) or hereditary sensory neuropathy type I (HSN I) loci. Neuromuscul. Disord. 12, 286-291 (2002).
5. Dawkins J. L., Hulme D. J., Brahmbhatt S. B., Auer-Grumbach M. and Nicholson G. A. Mutations in SPTLC1, encoding serine palmitoyltransferase, long chain base subunit-1, cause hereditary sensory neuropathy type I. Nat. Genet. 27, 309-312 (2001).
6. Bejaoui K. et al. SPTLC1 is mutated in hereditary sensory neuropathy, type 1. Nat. Genet. 27, 261-262 (2001).
7. Kwon J. M. et al. Assignment of a second Charcot-Marie-Tooth type II locus to chromosome 3q. Am. J. Hum. Genet. 57, 853-858 (1995).
8. De Jonghe P. et al. Mutilating neuropathic ulcerations in a chromosome 3q13-q22 linked Charcot-Marie-Tooth disease type 2B family. J. Neurol. Neurosurg. Psychiatry 62, 570-573 (1997).
9. Auer-Grumbach M. et al. Phenotype-genotype correlations in a CMT2B family with refined 3q13-q22 locus. Neurology 55, 1552-1557 (2000).
10. Echard A. et al. Interaction of a Golgi-associated kinesin-like protein with Rab6. Science 279, 580-585 (1998).
11. Nielsen E., Severin F., Backer J. M., Hyman, A. A. and Zerial, M. Rab5 regulates motility of early endosomes on microtubules. Nat. Cell. Biol. 1, 376-382 (1999).
12. Jordens I. et al. The RAB7 effector protein RILP controls lysosomal transport by inducing the recruitment of dynein-dynactin motors. Curr. Biol. 11, 1680-1685 (2001).
13. Cantalupo G., Alifano P., Roberti V., Bruni C. B. and Bucci C. Rab-interacting lysosomal protein (RILP): the RAB7 effector required for transport to lysosomes. EMBO. J. 20, 683-693 (2001).
14. Press B., Feng Y., Hoflack B. and Wandinger-Ness A. Mutant RAB7 causes the accumulation of cathepsin D and cation-independent mannose 6-phosphate receptor in an early endocytic compartment. J. Cell. Biol. 140, 1075-1089 (1998).
15. Lebrand C. et al. Late endosome motility depends on lipids via the small GTPase RAB7. EMBO. J. 21, 1289-1300 (2002).
16. Seabra M. C., Mules E. H. and Hume A. N. Rab GTPases, intracellular traffic and disease. Trends Mol. Med. 8, 23-30 (2002).
17. Tang B. L. Protein trafficking mechanisms associated with neurite outgrowth and polarized sorting in neurons. J. Neurochem. 79, 923-930 (2001).
18. Geppert M., Goda Y., Stevens C. F. and Sudhof T. C. The small GTP-binding protein Rab3A regulates a late step in synaptic vesicle fusion. Nature 387, 810-814 (1997).
19. Eggenschwiler J. T., Espinoza E. and Anderson K. V. Rab23 is an essential negative regulator of the mouse Sonic hedgehog signaling pathway. Nature 412, 194-198 (2001).
20. Elliott J. L., Kwon J. M., Goodfellow P. J. and Yee, W. C. Hereditary motor and sensory neuropathy IIB: Clinical and electrodiagnostic characteristics. Neurology 48, 23-28 (1997).

1-9. (canceled)

10. An isolated nucleic acid, or fragment thereof, encoding a polypeptide, wherein said isolated nucleic acid, or fragment thereof, encodes a dominant mutation in a RAB7 or ARHGEF10 polypeptide, and wherein the presence of said dominant mutation is indicative of a predisposition for, or the presence of, a peripheral neuropathy.

11. The isolated nucleic acid of claim 10, wherein the polypeptide is RAB7 gene product and wherein the peripheral neuropathy is Charcot-Marie-Tooth type 2B disease.

12. The isolated nucleic acid of claim 11, wherein said dominant mutation in RAB7 is Leu129Phe or Val162Met.

13. The isolated nucleic acid of claim 10, wherein the predisposition for, or the presence of, a peripheral neuropathy is a slowed nerve conduction velocity.

14. The isolated nucleic acid of claim 13, wherein said dominant mutation in ARHGEF10 is Thr109Ile or Asn704Ser.

15. The isolated nucleic acid, or fragment thereof, of claim 10, wherein said isolated nucleic acid, or fragment thereof, comprises a probe for detecting the presence of said dominant mutation indicative of a predisposition for, or the presence of, a peripheral neuropathy.

16. The isolated nucleic acid, or fragment thereof, of claim 10, wherein said isolated nucleic acid or fragment thereof is present in a vector.

17. The vector of claim 16, wherein the vector is present in a cell.

18. The vector of claim 17, where said vector is present in a non-human animal.

19. An isolated nucleic acid, or fragment thereof, of claim 10, wherein the dominant mutation comprises a dominant negative mutation.

20. A method for diagnosing the presence of, or predisposition for, a peripheral neuropathy in a human, comprising:
obtaining a biological sample from a human;
detecting the presence of a change in RAB7 or ARHGEF10, relative to wild-type, wherein the change in RAB7 or ARHGEF10 is indicative of a predisposition for, or presence of, a peripheral neuropathy.

21. The method according to claim 20, wherein detecting for the presence of a change in RAB7 or ARHGEF10 is selected from the group consisting of: a Southern blot assay, a Northern blot assay, a Western blot assay, a PCR assay, an immunoassay, amino acid analysis and a nucleic acid sequencing assay.

22. The method according to claim 20, wherein said change in RAB7 is indicative for a predisposition for, or presence of, Charcot-Marie-Tooth type 2B disease.

23. The method according to claim 22, wherein said change in RAB7 is Leu129Phe or Val162Met.

24. The method according to claim 20, wherein the predisposition for, or the presence of, a peripheral neuropathy is a slowed nerve conduction velocity.

25. The method according to claim 24, wherein said change in ARHGEF10 is Thr109Ile or Asn704Ser.

26. A diagnostic kit to detect the presence of, or predisposition for, a peripheral neuropathy in a human, comprising:
a means for detection of a change in a RAB7 or ARHGEF10 polypeptide or nucleic acid sequence, wherein the change in RAB7 or ARHGEF10 is indicative of a predisposition for, or the presence of, a peripheral neuropathy.

27. The diagnostic kit of claim 26, wherein the means for detection is selected from the group consisting of: a Southern blot assay, a Northern blot assay, a Western blot assay, a PCR assay, an immunoassay, amino acid analysis and a nucleic acid sequencing assay.
